SEQUENCE LISTING (6 sequences)

SEQ ID NO: 1 - protein, 30 aa, Artificial Sequence
  Synthetic construct
EVEALEKKVAALECKVQALEKKVEALEHGW

SEQ ID NO: 2 - protein, 30 aa, Artificial Sequence
  Synthetic construct
GGGEIWKLHEEFLCKFEELLKLHEERLKKM

SEQ ID NO: 3 - protein, 12 aa, Artificial Sequence
  Synthetic construct
AYSSGAPPMPPF

SEQ ID NO: 4 - protein, 30 aa, Artificial Sequene
  Synthetic constructSynthetic construct
EGKAGEKAGAALKCGVQELEKGAEAGEGGW

SEQ ID NO: 5 - protein, 30 aa, Artificial Sequence
  Synthetic construct
EVEALEKKVAALESKVQALEKKVEALEHGW

SEQ ID NO: 6 - protein, 34 aa, Artificial Sequence
  Synthetic construct
EVEALEKKVAALECKVQALEKKVEALEHGWGGGK

(19) United States
(12) Patent Application Publication
(10) Pub. No.: US 2017/0165382 A1
(43) Pub. Date: Jun. 15, 2017

(54) NANOCARRIERS FOR CANCER TREATMENT

(71) Applicant: The Regents of the University of California, Oakland, CA (US)

(72) Inventors: Ting Xu, Berkeley, CA (US); John Forsayeth, San Francisco, CA (US); Katherine Ferrara, Davis, CA (US)

(21) Appl. No.: 15/349,946

(22) Filed: Nov. 11, 2016

Related U.S. Application Data
(60) Provisional application No. 62/254,508, filed on Nov. 12, 2015.

(51) Int. Cl. A61K 51/08; A61K 31/704; A61K 51/12

(52) U.S. Cl. CPC A61K 51/088; A61K 51/1234; A61K 31/704

(57) ABSTRACT

The present invention provides conjugates containing metal binding ligands, as well as nanocarriers prepared from the conjugates.

### CROSS-REFERENCES TO RELATED APPLICATIONS

The present application claims priority to U.S. Provisional Pat. Appl. No. 62/254,508, filed Nov. 12, 2015, which application is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. NIHR01CA103828, R01CA134659, and R21EB016947. The Government has certain rights in this invention.

### BACKGROUND OF THE INVENTION

Glioblastoma multiforme (GBM) is the most common and aggressive malignant primary brain tumor, with a median patient survival of 12-15 months [1-3]. Combining radiotherapy and post-surgical chemotherapy using cisplatin [4, 5], irinotecan [6-8], thalidomide [9, 10], or bevacizumab [11, 12] has only led to a limited improvement in survival rate [13, 14]. The blood-brain barrier (BBB) typically limits the accumulation of therapeutics within the brain and such drugs can be deactivated by intra- and extracellular enzymes in the BBB. The BBB includes a range of passive and active transport mechanisms: 1) a paracellular pathway, regulated by tight junctions; 2) a lipophilic pathway, through the lipid membranes; 3) specific receptor-mediated transcytosis actuated by specific interactions with receptors on cerebral endothelial cells; and 4) non-specific adsorptive-mediated transcytosis, triggered by interactions between positively-charged species and negatively-charged lipid membranes on endothelial cells [15]. The BBB is altered in the presence of diseases such as GBM and transport of nanotherapies is enhanced through junctions that are altered by the presence of disease. However, while essentially all GBM patients have significant BBB disruption, the disruption is variable across the tumor and GBM patients also have regions of tumor with limited BBB permeability [16]. Therefore, the development of strategies to enhance drug accumulation is important. Further, when drugs are delivered to the GBM tumor parenchyma, efflux transporters actively pump the drug out of the target cell [15, 17-19]. GBM therapeutics must be administered at a high dose that can lead to severe side effects and early termination of treatment, and thus, there is an urgent need to develop nanocarriers for the treatment of GBM.

It is well known that the surface chemistry of a nanocarrier determines its pharmacokinetics (PK), biodistribution and clearance pathway [20], and PEGylation is required to avoid recognition by the reticuloendothelial system (RES) and extend the circulation time. However, in the absence of additional surface modification, PEGylated nanocarriers typically do not cross the BBB [21, 22]. When the BBB is comprised by disease, passive delivery of nanotherapeutics is feasible. Passive delivery of long-circulating nanoparticles via the enhanced permeability retention (EPR) effect has been the major mechanism for nanoparticle uptake into tumors [23-27]. In general, smaller nanoparticles (15˜50 nm) demonstrate a greater EPR effect and intratumoral distribution than larger nanoparticles (100˜300 nm) and therefore show the potential to enhance accumulation [28, 29]. However, systematic studies of the effect of nanocarrier size and surface chemistry on the carrier's ability to accumulate within GBM tumor tissue have been lacking. Previous studies have shown that the vascular permeability increases in highly angiogenic glioblastoma due to the disrupted BBB providing a conduit for the delivery of nanotherapies [30-32]. However, the vascular permeability is reduced in brain tumors as compared with tumors within other organs and the size limit for nanoparticles observed to preferentially accumulate in glioblastoma (7-100 nm) is smaller than that in colorectal carcinoma, heptoma, and sarcoma (380-2000 nm) [33]. Once localized in the tumor, there is increasing evidence that nanocarriers need to be below a certain size to achieve significant tumor penetration [34-37].

Enhanced delivery to brain tumors with small nanoparticles has not yet been experimentally validated. Hobbs et al. demonstrated that particle permeability for orthotopic brain tumors was limited to particles with a diameter ranging from 7 to 100 nm [33]; however, differences within the size range were not described. Kim et al. reported that PEGylated silica nanoparticle uptake in a U87MG mouse xenograft was greater with 100-150 nm particles as compared with larger and smaller particles (40 and >300 nm) [38]. However, in this study the tumor was implanted in the mouse shoulder, which may differ in the pore cutoff size as compared with the orthotopic brain tumor.

The study compares the accumulation of two ⁶⁴Cu-labeled nanocarriers: a PEGylated 110-nm liposome with similar pharmacokinetics to other long-circulating liposomes [39] and recently developed 20 nm 3-helix micelles (3HM) [40]. This family of highly stable, long circulating 3HM is based on a coiled-coil protein tertiary structure that is routinely used to present ligand clusters on the cell surface. 3HM is based on a peptide-polymer conjugate amphiphile schematically shown in FIG. 1. The headgroup of the amphiphile consists of a peptide that self-associates to form a coiled-coil 3-helix bundle and a PEG chain (2000 Da) attached to the exterior of 3-helix bundle at the middle position. A short PEG chain (750 Da) is attached to one end of the peptide (c-terminus) and act as stealth layer on the surface of micelle. The hydrophobic portion of the amphiphile is a double alkyl tail attached to the other end of the peptide (N-terminus). The amphiphile can be readily synthesized at high purity. Once dissolved in aqueous solution, it self-assembles to form 3HM that is ˜20 nm in size with very low polydispersity in size [40-44]. Systematic characterization confirmed very slow subunit exchange kinetics and excellent kinetic stability of the micelle under physiological condition. In vivo studies further showed that the 3HM has a circulation half-life of 29.5 hours with minimal accumulation in the liver and spleen[40].

We have previously developed methods to label liposomes and micelles with ⁶⁴Cu- and have shown these labels to be stable in serum over 48 hours [39]. The use of same chelator, 6-BAT, on micelles retained ⁶⁴Cu in micelles and resulted in long circulation (circulation half-life: 29.5 h) in plasma fraction over blood at 48 hours. Further, the stability of labeled micelles in BSA, observed by time-resolved FRET, demonstrated a trace level dissociation from micelles over 24 hours [40]. Using co-registered positron emission tomography (PET) and magnetic resonance (MR) images, here we performed systematic studies to investigate how the nanocarrier's size affects the pharmacokinetics and bio distribution in rodents with GBM xenograft. The resulting data suggest that imaging of nanoparticle distribution and tumor kinetics can be used to improve the design of nanoparticles for GBM treatment and confirm that GBM delivery can be improved with small nanocarriers.

### BRIEF SUMMARY OF THE INVENTION

Applying methods for the synthesis of stable particles and PET labeling demonstrated in previous studies, here, we explore the accumulation of long-circulating liposomes and 3HM in glioblastoma using ⁶⁴Cu-labeled drug carriers and the combination of PET and MM. The PEGylation on the surface of the carriers provided a similar charge and facilitated studies of the enhanced permeability and retention of nanoparticles based on differences in their diameters. Although previous studies have demonstrated that vascular permeability is reduced in brain tumors compared to tumors within other organs, enhanced delivery to brain tumors with small nanoparticles has not been clearly demonstrated. Here, we demonstrate that the uptake of 20-nm 3HM is significantly greater than 110-nm liposomes in glioblastoma 7 hours after injection (FIG. 6c). Importantly, we observed that the micelles continued to accumulate over the period studied here, and therefore these small particles were not clearing from the lesion, even in the absence of a targeting moiety. The micelles were well distributed throughout the tumor, potentially providing an opportunity to effectively treat disease when a drug or radiotherapy is attached.

The average fold increase for liposome and 3HM accumulation in glioblastoma compared to background (left striatum) was 2.78 and 5.12, respectively (FIG. 6d). Although those values were lower than those measured for human glioblastoma, which has 13˜19-fold higher accumulation of stealth liposomes vs. normal brain [52], the overall results demonstrate that liposomes and micelles enhanced accumulation in glioblastoma.

From the bio distribution data obtained after perfusion (FIG. 7), the greater accumulation associated with a greater EPR effect in an advanced xenograft (>100 mm³) was confirmed. Micelle accumulation was greater than that of liposomes regardless of the progression of the xenograft (FIG. 7a). Although the radioactivity in the left brain was ˜10-fold lower than in the right brain (FIG. 7b), the accumulation in the normal left brain showed two significant effects associated with the adjacent disease. First, in the contralateral left brain, 3HM uptake increased with xenograft progression in the implanted right brain. The permeability of the contralateral brain could be affected by the pressure induced by the growing tumor or by cytokines and growth factors associated within tumor [53]. Second, the 110-nm liposomal uptake in the left brain was similar (˜0.008% ID/g) regardless of the glioblastoma diameter. Thus, the extravasation of 110-nm liposomes was limited by the vascular pore size cutoff but relatively small 20-nm micelles crossed the BBB.

A major advantage of the PET-MRI techniques applied here is the opportunity to simultaneously estimate the PK and the local blood volume. Extended circulation of nanoparticles in the blood is crucial for the extravasation through leaky vasculature and accumulation in tumors. In our previous PK studies of liposomes and micelles in a mouse model [40, 54], the half-life of ⁶⁴Cu-liposomes and ⁶⁴Cu-micelles was 18 and 25 h (one-phase decay), respectively. Here, we observed a shorter half-life for both particles in blood (t_{1/2 liposomes and micelles}=16.5 and 15.5 h). The observed circulation time was longer than ^{99m}Tc-labeled HYNIC-PEG liposomes previously studied in a rat model where only 52% ID remained in the blood pool 4 h after injection [55]. We assume that the reduced half-life observed here was due to differences in the vascular physiology between the two species. Here, the similar blood clearance of the nanoparticles in blood facilitated a direct comparison of the radioactivity in the tissues at the same time point.

When evaluating long-circulating nanoparticles, the blood volume can also be estimated by evaluating the radioactivity in the blood and tumor at the time of injection as calculated by a previously described radiometric method [51]. Previous MR studies in the rat brain reported a relationship between blood volume and vessel size where approximately 15% of C6 gliomas demonstrated an increased cerebral blood volume as compared to gray matter, and 90% demonstrated an increased average vessel size [50]. In a subsequent study, no correlation was found between blood vessel density and tumor progression in GBM [56]. Here, we observed a 62-82% increase in the % vascular volume in the tumor as compared to the contralateral LBV (FIG. 6a) but the % vascular volume was not significantly different between small (<100 mm³) and large tumors (>100 mm³) (FIG. 6b). The vascular volume in the adjacent left brain also was not significantly changed with xenograft progression (FIG. S4). Immunohistochemistry (IHC) with a CD31 antibody demonstrated larger vessels (FIG. 8a, black arrow) in glioblastoma lesions, which were not observed in normal brain tissue (striatum, FIG. 8b, black arrow). Previous work also demonstrated large vessels in tumors larger than 4 mm [57]. In addition, in our study MR images (arrows in FIG. 3 and FIG. 4a-b) resolved large vessels within glioblastoma lesions. The TBV and LBV results suggest that vascularization of glioblastoma increases the vascular volume in glioblastoma.

The biodistribution of both nanoparticles in organs such as the heart, lung, stomach, muscle, bone, liver and kidney was similar. As we observed in our previous study [40], the micelle accumulation was significantly lower in spleen than that observed with liposomes, which could ultimately reduce the treatment toxicity.

Recently, 3HM micelles were loaded with doxorubicin and prolonged drug bioavailability in circulation [42, 43], which may improve therapeutic efficacy and reduce splenic toxicity. Success in ongoing research with respect to loading or conjugating anticancer drugs to micelles could provide a promising method to treat glioblastoma [58, 59].

In conclusion, current GBM treatment includes invasive surgery, radiotherapy, and chemotherapy; however, drug delivery remains a major challenge. Here, we demonstrated that 3HM accumulate within glioblastoma to a significantly greater extent than 110-nm liposomes. PET/MR co-registration of brain images with multiple imaging modalities may facilitate the monitoring of disease progression and planning of treatment regimens.

### BRIEF DESCRIPTION OF THE DRAWINGS

FIG. 1 shows a schematic figure of the experimental procedure, which compares the accumulation of liposomes and micelles within glioblastoma multiforme in the rat brain. Polyethylene glycol (PEG) is shown on the surface of the nanoparticles (dC18: distearoyl lipid, 6-BAT: 6-aminobenzylTETA). Nanoparticles were intravenously injected through the tail vein.

FIG. 2 shows solution SAXS results comparing micelles with and without PEG750 layer on the exterior of the micelle. The lines indicate best fit to the core-shell model. Data for dC18-1CW(P2k) has been offset vertically for clarity.

FIG. 3 shows coregistered PET/MR images (upper) and MR only images (lower) of rat brain at 21 h post-injection of ⁶⁴Cu-liposomes and ⁶⁴Cu-micelles. Arrows indicate developed blood vessels in glioblastoma. The maximum and minimum shading scale from PET images represent 1 and 0% ID/cc, respectively and the size of the white scale bars in image is 2 mm.

FIG. 4a shows coregistered PET/MR images of rat brain post injection of ⁶⁴Cu-liposomes (upper row) and ⁶⁴Cu-micelles (lower row). From left to right, PET/MR images are acquired at 0, 3.5, 7.5 and 21 h after injection. Each image represents a 1 mm thick slice image of the glioblastoma lesion. Arrows indicate blood vessels. Maximum and minimum values of the shading scale are 1.0 and 0.3% ID/cc, respectively.

FIG. 4b shows coregistered PET/MR images of rat brain post injection of ⁶⁴Cu-liposomes (upper row) and ⁶⁴Cu-micelles (lower row). PET/MR images acquired at 21 hours after injection, from left (posterior) to right (anterior). Each image represents a 1 mm thick slice image of the glioblastoma lesion. Arrows indicate blood vessels. Maximum and minimum values of the shading scale are 1.0 and 0.3% ID/cc, respectively.

FIG. 5A shows the blood clearance of ⁶⁴Cu-liposomes (black circle) and ⁶⁴Cu-micelles (gray circle) obtained from ROI analyses at 0, 3.5, 7 and 21 h post-injection. Curve was fit with a one phase decay (Yₗᵢₚₒₛₒₘₑₛ=6.104exp^{-0.04206×}(R²=0.8330) and Y_{micelles}=6.432exp^{-0.04461×}(R²=0.8167).

FIG. 5B shows the radioactivity (% ID/g) of liposomes (black bar) and micelles (gray bar) in blood at 22 h post-injection.

FIG. 6A shows the quantification of liposomes (n=6) and micelles (n=5) in glioblastoma, obtained from ROI analysis (glioblastoma) of PET/MR images. Tumor blood volume (TBV) and contralateral left brain blood volume (LBV) calculated by ROI analysis of glioblastoma (right brain) and contralateral striatum (left brain) from ⁶⁴Cu-liposome (black bar) and -micelle (gray bar) injected rats. Statistical significance was determined by two-way ANOVA analysis corrected by Sidak's multiple comparison, significance: *=P<0.05, **=P<0.01, ***=P<0.001, ****=P<0.0001).

FIG. 6B shows the quantification of liposomes (n=6) and micelles (n=5) in glioblastoma, obtained from ROI analysis (glioblastoma) of PET/MR images. Comparison of % vascular volume between two groups with different size of glioblastoma.

FIG. 6C shows the quantification of liposomes (n=6) and micelles (n=5) in glioblastoma, obtained from ROI analysis (glioblastoma) of PET/MR images. Blood radioactivity subtracted time activity curves of liposomes (round with dashed line) and micelles (square with dotted line). Data points represent 0, 3.5, 7, and 21 h post-injection. Statistical significance was determined by two-way ANOVA analysis corrected by Sidak's multiple comparison test, significance: *=P<0.05, **=P<0.01, ***=P<0.001, ****=P<0.0001).

FIG. 6D shows the quantification of liposomes (n=6) and micelles (n=5) in glioblastoma, obtained from ROI analysis (glioblastoma) of PET/MR images. Glioblastoma-to-background (BG) ratio of liposomes (black) and micelles (gray) in glioblastoma over contralateral left striatum, obtained from PET/MR images with blood radioactivity at 21 h Statistical significance was determined by an unpaired t test with Welch's correction, significance: *=P<0.05, **=P<0.01, ***=P<0.001, ****=P<0.0001).

FIG. 7A shows the bio distribution of ⁶⁴Cu-liposomes (black bar) and -micelles (gray bar) in the right brain, where the right brain bears glioblastoma. Percent injected dose per gram (% ID/g) was obtained after perfusion of blood at 22 h post-injection of ⁶⁴Cu-liposomes (n=6) and -micelles (n=5). Right bar graphs are differentiated by tumor size. Statistical significance of was determined by two-way ANOVA analysis corrected by Sidak's multiple comparison test, significance: *=P<0.05, **=P<0.01, ***=P<0.001, ****=P<0.0001).

FIG. 7B shows the bio distribution of ⁶⁴Cu-liposomes (black bar) and -micelles (gray bar) in the left brain, where the right brain bears glioblastoma. Percent injected dose per gram (% ID/g) was obtained after perfusion of blood at 22 h post-injection of ⁶⁴Cu-liposomes (n=6) and -micelles (n=5). Right bar graphs are differentiated by tumor size. Statistical significance was determined by two-way ANOVA analysis corrected by Sidak's multiple comparison test, significance: *=P<0.05, **=P<0.01, ***=P<0.001, ****=P<0.0001).

FIG. 7C shows the bio distribution of ⁶⁴Cu-liposomes (black bar, n=6) and ⁶⁴Cu-micelles (gray bar, n=5) at 22 h post-injection. Statistical significance was determined by unpaired t test with Welch's correction, significance: *=P<0.05, **=P<0.01, ***=P<0.001, ****=P<0.0001).

FIG. 8A shows the immunohistochemistry of glioblastoma-bearing right brain. Upper and lower images are from H&E and CD31, respectively. (CC: cerebral cortex, STR: striatum, T: tumor, and V: vessel).

FIG. 8B shows the immunohistochemistry of contralateral left brain. Upper and lower images are from H&E and CD31, respectively. (CC: cerebral cortex, STR: striatum, T: tumor, and V: vessel).

FIG. 9 shows the helix wheel of a typical de novo designed three-helix bundle-forming peptide.

### DETAILED DESCRIPTION OF THE INVENTION

### I. General

The present invention provides micelle nanocarriers for in vivo delivery of drugs and other cargo, where the nanocarriers are modified with a metal binding ligand. The nanoparticles can be targeted or untargeted. Suitable cargo that can be delivered by the nanocarriers of the present invention include, but are not limited to, vaccines, nucleic acids such as DNA or RNA, peptides, proteins, imaging agents, and drugs. The nanoparticles of the present invention are also useful for gene therapy, the administration of an expressed or expressible nucleic acid to a subject.

The nanocarriers are composed of metal binding ligand lipid-peptide conjugates that self-assemble to form the micelles, and are capable of binding to metals for imaging or therapeutic purposes. The conjugates include a hydrophobic block and headgroup containing a helical peptide, a polymer block, and a metal binding ligand. Helix bundle formation by the peptides results in alignment of the hydrophobic block at the N-terminal end of the peptide bundle, with the polymer block covalently linked to the peptide along the length of the peptide, and the metal binding ligand covalently linked to the C-terminal end of the peptide. The micelles resulting from conjugate assembly contain a polymer shell on the micelle surface. The surface C-terminal polymer, in particular, contributes to the surprising stability and long circulation time of the micelle nanoparticles, as compared to micelles assembled from conjugates without a C-terminal polymer and other previously known self-assembled nanocarrier structures.

### II. Definitions

“Conjugate” refers to a compound having a first polymer, a metal binding ligand, a peptide and a hydrophobic moiety all linked together. The conjugates are capable of self-assembling to form helix bundles. The helix bundles include from 2 to 6 conjugates, typically 3 or 4.

“Polypeptide,” “peptide,” and “protein” are used interchangeably herein to refer to a polymer of amino acid residues. All three terms apply to amino acid polymers in which one or more amino acid residues is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds. The peptides of the present invention can be helical in structure and form a coiled-coil tertiary protein structure. The formation of coiled-coil tertiary structure provides a structural scaffold to position conjugated polymers and define the shape of individual sub-units for the nanoparticle. The helices also enhance the rigidity of the sub-unit and enable the geometric packing in a manner similar to that of virus particles.

“N-terminus” refers to the first amino acid residue in a protein or polypeptide sequence. The N-terminal residue contains a free α-amino group.

“C-terminus” refers to the last amino acid residue in a protein or polypeptide sequence. The C-terminal residue contains a free carboxylate group.

“Polymer” refers to a macromolecule having repeating units connected by covalent bonds. Polymers can be hydrophilic, hydrophobic or amphiphilic. Hydrophilic polymers are substantially miscible with water and include, but are not limited to, polyethylene glycol. Hydrophobic polymers are substantially immiscible with water and include, but are not limited to, polybutadiene and polystyrene. Amphiphilic polymers have both hydrophilic and hydrophobic properties and are typically block copolymers of a hydrophilic and a hydrophobic polymer. Polymers include homopolymers, random copolymers, and block copolymers. Specific polymers useful in the present invention include polyethylene glycol, N-isopropylacrylamide (NIPAM), polybutadiene and polystyrene, among others.

“Metal binding ligand” includes any ligand capable of chelating to a metal. Representative metal binding ligands include 6-aminobenzyl TETA (1,4,8,11-tetraazacyclotetradecane-1,4,8,11-tetraacetic acid) for binding to copper.

“Hydrophobic moiety” refers to polymers or small molecules that are hydrophobic. Examples of hydrophobic moieties include, but are not limited to, hydrophobic polymers such as polybutadiene and polystyrene, as well as the lipid moieties of the present invention.

“Lipid moiety” refers to a moiety having at least one lipid. Lipids are small molecules having hydrophobic or amphiphilic properties and are useful for preparation of vesicles, micelles and liposomes. Lipids include, but are not limited to, fats, waxes, fatty acids, cholesterol, phospholipids, monoglycerides, diglycerides and triglycerides. The fatty acids can be saturated, mono-unsaturated or poly-unsaturated. Examples of fatty acids include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). The lipid moiety can include several fatty acid groups using branching groups such as lysine and other branched amines.

“Alkyl” refers to a straight or branched, saturated, aliphatic radical having the number of carbon atoms indicated. Alkyl groups can have up to 24 carbons atoms and include heptyl, octyl, nonyl, decyl, dodecyl, tridecyl, tetradecyl, pentadecyl, hexadecyl, heptadecyl, octadecyl, nonadecyl, icosyl, and the like. Alkyl can include any number of carbons such as C₆₋₂₀, C₆₋₁₈, C₆₋₁₆, C₈₋₂₄, C₈₋₂₂, and C₈₋₂₀. Alkyl groups can be substituted with substituents including fluorine groups.

“Acyl” refers to a carbonyl radical (i.e., C═O) substituted with an alkyl group as defined above. The number of carbon atoms indicated for an acyl group includes the carbonyl carbon and the alkyl carbons. Acyl groups can have up to 24 carbons atoms and include heptoyl, octoyl, nonoyl, decoyl, dodecoyl, tridecoyl, tetradecoyl, pentadecoyl, hexadecoyl, heptadecoyl, octadecoyl, nonadecoyl, icosoyl, and the like. Acyl can include any number of carbons such as C₆₋₂₀, C₆₋₁₈, C₆₋₁₆, C₈₋₂₄, C₈₋₂₂, and C₈₋₂₀. Acyl groups can be substituted with substituents including fluorine groups.

“Anthracycline” refers to natural products of Streptomyces peucetius and related derivatives. Anthracyclines are glycosides containing an amino sugar and a fused, tetracyclic aglycone. Many anthracyclines demonstrate antibiotic and antineoplastic activity. Examples of anthracyclines include, but are not limited to, daunorubicin, doxorubicin, epirubicin, and idarubicin.

“Macrolide” refers to compounds characterized by a large (typically 14-to-16-membered) lactone ring substituted with pendant deoxy sugars. Many macrolides demonstrate antibiotic and immunomodulatory activity. Examples of macrolides include, but are not limited to, rapamycin, clarithromycin, and erythromycin.

“Therapeutic agent” refers to an agent capable of treating and/or ameliorating a condition or disease. Therapeutic agents include, but are not limited to, compounds, drugs, peptides, oligonucleotides, DNA, antibodies, and others.

“Diagnostic agent” refers to an agent capable of diagnosing a condition or disease. Diagnostic agents include, but are not limited to, dyes and radiolabels.

“Nucleic acid,” “oligonucleotide,” and “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. The term nucleic acid is used interchangeably with gene, cDNA, and mRNA encoded by a gene.

“Contacting” refers to the process of bringing into contact at least two distinct species such that they can interact. In some cases, such interactions include non-covalent interactions such as ionic interactions and van der Waals interactions. In some cases, the interaction results in a covalent bond-forming reaction. In these cases, it should be appreciated that the resulting reaction product can be produced directly from a reaction between the added reagents or from an intermediate from one or more of the added reagents which can be produced in the reaction mixture.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

“Amino acid analogs” refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, ornithine, pentylglycine, pipecolic acid and thioproline.

“Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Exemplified hydrophobic amino acids include valine, leucine, isoleucine, methionine, phenylalanine, and tryptophan. Exemplified aromatic amino acids include phenylalanine, tyrosine and tryptophan. Exemplified aliphatic amino acids include serine and threonine. Exemplified basic amino acids include lysine, arginine and histidine. Exemplified amino acids with carboxylate side-chains include aspartate and glutamate. Exemplified amino acids with carboxamide side chains include asparagines and glutamine. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Helix bundle” refers to a structure formed by the self-assembly of a plurality of conjugates of the present invention, where the hydrophobic moieties are aligned with each other at one end of the peptide bundle (typically the N-terminal end) and the polymers of each conjugate are arranged along the length of the peptide bundle and at the end of the peptide bundle opposite the hydrophobic moieties (typically the C-terminal end).

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Treat”, “treating,” and “treatment” refer to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to a patient or subject; decreasing the frequency or duration of the symptom or condition; or, in some situations, preventing the onset of the symptom or condition. The treatment or amelioration of symptoms can be based on any objective or subjective parameter; including, e.g., the result of a physical examination.

“Cancer” includes solid tumors and hematological malignancies. Cancer includes but is not limited to cancers such as carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, brain and central nervous system, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma (see, CANCER: PRINCIPLES AND PRACTICE (DeVita, V. T. et al. eds 1997) for additional cancers). One of skill in the art will appreciate that other cancers and proliferative disorders can be treated by the particles of the present invention.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

### III. Conjugates, Helix Bundles, and Particles

In some embodiments, the present invention provides a conjugate having a first peptide with from about 10 to about 100 amino acids, wherein the peptide adopts a helical structure. The conjugate also includes: a first polymer covalently linked to an amino acid residue of the peptide, other than the N-terminal and C-terminal residues; a metal binding ligand covalently linked to the C-terminal amino acid residue of the peptide; and a hydrophobic moiety covalently linked to the N-terminus of the peptide, wherein the hydrophobic moiety comprises a third polymer or a lipid moiety.

Peptides useful in the conjugates of the present invention are those that adopt a helical conformation. The peptides can be of any suitable length, such as from about 10 to about 1000 amino acids, or from about 10 to about 500 amino acids, or from about 10 to about 100 amino acids. In some embodiments, the peptide can be SEQ ID NO: 1, SEQ ID NO: 2, SEQ ID NO: 4, SEQ ID NO: 5, and SEQ ID NO: 6.

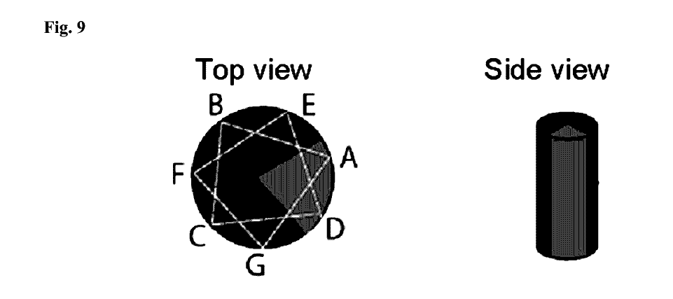

In a preferred embodiment, the first peptide can self-associate to form tertiary peptide structures. FIG. 9 shows the helix wheel of a typical de novo designed helix bundle. The peptide primary structure is characterized by a heptad periodicity, -abcdefg-. The peptide is amphipathic, forming a helix having a hydrophobic face and a hydrophilic face. Helix bundle formation is driven by the hydrophobic interactions between amino acids at positions a and d of each helix, forming a hydrophobic core. The bundle is further stabilized by the salt bridges between amino acids at positions e and g of adjacent helices. Accordingly, some embodiments of the invention provide conjugates wherein the peptide is characterized by a heptad periodicity -abcdefg-. The peptide can contain, for example, one heptad, two heptads, three heptads, four heptads, five heptads, six heptads, seven heptads, nine heptads, ten heptads, eleven heptads, or twelve heptads. Amino acids at positions a and d will generally be hydrophobic amino acids (e.g., valine, leucine, isoleucine, methionine, phenylalanine, tryptophan, or a conservative substitution thereof), although different hydrophobic amino acids can be present at each a position and/or each d position. Amino acids at positions e and g will generally be basic or acidic amino acids (e.g., lysine, arginine, histidine, aspartate, glutamate, or a conservative substitution thereof), although different basic or acidic amino acids can be present at each e position and/or each g position. In certain embodiments, glycine can be present at one e position or one g position. In certain embodiments, glycine can be present at up to three e positions or up to three g positions. The peptide can further include up to five amino acids on either end of the heptad repeats. In some embodiments, the peptide further includes one amino acid on each end of the heptad repeats (i.e., one additional amino acid at the C-terminus and one additional amino acid at the N-terminus of the peptide).

In some embodiments, the peptide contains from four to seven heptad repeats having isoleucine in each a position and leucine in each d position, and the peptide assembles to form dimeric helix bundles. In some such embodiments, the peptide has glutamic acid or aspartic acid at each e position. In some such embodiments, the peptide has lysine at each g position. In some such embodiments, amino acids at the b, c, and f positions are independently selected from the group consisting of alanine, aspartic acid, cysteine, glutamine, histidine, and lysine. In certain embodiments, glycine can be present at one e position or one g position. The peptides in the dimeric helix bundle can assemble such that both C-termini are arranged at the same end of the helix bundle, or such that both C-termini are arranged at opposite ends of the helix bundle.

In some embodiments, the peptide contains from four to seven heptad repeats having leucine in each a position and isoleucine in each d position, and the peptide assembles to form tetrameric helix bundles. In some such embodiments, the peptide has glutamic acid or aspartic acid at each e position. In some such embodiments, the peptide has lysine at each g position. In some such embodiments, amino acids at the b, c, and f positions are independently selected from the group consisting of alanine, aspartic acid, cysteine, glutamine, histidine, and lysine. In certain embodiments, glycine can be present at one e position or one g position. The peptides in the tetrameric helix bundle can assemble such that all C-termini are arranged at the same end of the helix bundle; such that two of the C-termini are arranged at one end of the helix bundle and two of the C-termini are arranged at the opposite end of the helix bundle; or such that one or three of the C-termini are arranged at one end of the helix bundle and the remaining C-termini are arranged at the opposite end of the helix bundle.

In some embodiments, the peptide contains from four to seven heptad repeats having leucine, isoleucine, or valine in each a position and each d position, and the peptide assembles to form trimeric helix bundles. In some such embodiments, the peptide has glutamic acid or aspartic acid at each e position. In some such embodiments, the peptide has lysine at each g position. In some such embodiments, amino acids at the b, c, and f positions are independently selected from the group consisting of alanine, aspartic acid, cysteine, glutamine, histidine, and lysine. In some such embodiments, amino acids at the b, c, and f positions are independently selected from the group consisting of alanine, aspartic acid, cysteine, glutamine, histidine, and lysine. In certain embodiments, glycine can be present at one e position or one g position. The peptides in the trimeric helix bundle can assemble such that all C-termini are arranged at the same end of the helix bundle, or such that two of the C-termini are arranged at one end of the helix bundle and the third C-terminus is arranged at the opposite end of the helix bundle.

The helicity of a particular peptide sequence can be measured using standard biochemical techniques known to those of skill in the art. Such techniques include, but are not limited to, circular dichroism (CD), thermal denaturation, and equilibrium centrifugation. CD measurements can be made using a spectropolarimeter (e.g., a Jasco J810 spectropolarimeter). CD spectra can be collected, for example, from 260 to 190 nm at 0.2 nm intervals, a rate of a 100 nm/min, a response time of 4 s, and a bandwidth of 1 nm. Temperature melt curves can be measured using any appropriate peptide concentration (e.g., 200 μM) and any suitable temperature range (e.g., 4° C. to 100° C.). As a non-limiting example, the ellipticity can be monitored at 222 nm as the temperature is increased from 5° to 95° C. in 5° C. increments at a rate of 1° C./min, with a 1 min equilibration time at each temperature before the measurement is taken. One hundred percent helicity is estimated using the formula:

[θ]₂₂₂40,000\check{G}[1−(2.5/n)].

In some embodiments, the first peptide can be a de novo designed 3-helix bundle peptide, such as, but not limited to, SEQ ID NO: 1. In some embodiments, 1-50 amino acids can be appended to the C-terminus of the first peptide without interfering with micelle formation. In some embodiments, 1-25 amino acids, preferably 1-10 amino acids and more preferably 1-5 amino acids, can be appended to the C-terminus of the first peptide. In some embodiments, the first peptide sequence can be a control peptide sequence that a forms random coil such as, but not limited to, SEQ ID NO: 4. In some embodiments, the first peptide can be designed based on SEQ ID NO:5, and have similar characteristics including PI and hydrophobicity. In some embodiments, the first peptide sequence can be a heme-binding peptide that is able to form 4-helix bundles such as SEQ ID NO: 2.

The conjugates of the present invention also include a first polymer and a metal binding ligand. The first polymer can be any suitable polymer. Exemplary polymers include hydrophilic, hydrophobic and amphiphilic polymers. As a non-limiting example, the first polymer can be independently selected from polyethylene glycol (PEG or P), poly(N-isopropylacrylamide) (NIPAM), polybutadiene (PBD), and polystyrene (PS). In some embodiments, the first polymer include hydrophilic polymers. Hydrophilic polymers are miscible with water, and include, but are not limited to, polyethylene glycol, NIPAM, and cellulose. In some embodiments, the first polymer include polyethylene glycol.

The first polymer can be linked to any point of the peptide other than the N-terminal amino acid residue and the C-terminal amino acid residue. Any suitable covalent linkage is useful for attaching the first polymer to the peptide. For example, the covalent linkage can be via an ester, amide, ether, thioether or carbon linkage. In some embodiments, the first polymer can be modified with a maleimide that reacts with a sulfhydryl group of the peptide, such as on a cysteine. In some embodiments, the first polymer can be linked to the peptide via click chemistry, by reaction of an azide and an alkyne to form a triazole ring.

In general, the metal binding is linked to the C-terminal amino acid residue of the polymer. The metal binding ligand can be any ligand suitable for chelating a metal. For example, the metal binding ligand can be 6-aminobenzyl TETA ((1,4,8,11-tetraazacyclotetradecane-1,4,8,11-tetraacetic acid) for binding to copper. In some embodiments, the metal binding ligand is a copper binding ligand. In some embodiments, the copper binding ligand includes 6-aminobenzyl TETA.

Conjugate assembly properties, as well as the stability of conjugate bundles and micelles, depend in part on conjugate architecture and the molecular weight of the polymers in the conjugate. The shape of a conjugate will influence the size and shape of the micelle resulting from conjugate assembly. The molecular weight of the first polymer can be chosen so as to tune the assembly and stability of the micelles. In general, polymer molecular weights are sufficiently large to stabilize the assembled micelles but not so large as to interfere with helix bundle assembly and micelle assembly. In some embodiments, the molecular weight of the first polymer can be from about 500 Da to about 10,000 Da. In some embodiments, the molecular weight of the first polymer can be, for example, from about 1000 Da to about 7500 Da, or from about 2000 Da to about 5000 Da. The molecular weight of the first polymer can be about 500 Da, or about 1000 Da, or about 2000 Da, or about 3000 Da, or about 4000 Da, or about 5000 Da, or about 6000 Da, or about 7000 Da, or about 8000 Da, or about 9000 Da, or about 10,000 Da. In some embodiments, the molecular weight of the first polymer can be from about 1000 Da to about 5000 Da. In some embodiments, the molecular weight of the first polymer can be about 2000 Da.

In some embodiments, the hydrophobic moiety can be a second polymer. Polymers useful as the hydrophobic moiety include hydrophobic polymers such as polybutadiene, polystyrene, polyacrylates, polymethacrylates, polydiacetylene, and the like. In some embodiments, the hydrophobic moiety can be polybutadiene. In some embodiments, the second polymer can be from about 1000 Da to about 3000 Da. In some embodiments, the second polymer can be from about 1100 Da to about 2600 Da. In some embodiments, the second polymer can be from about 1000 Da to about 2000 Da.

In some embodiments, the hydrophobic moiety can be a lipid moiety. Lipid moieties useful in the present invention include from 1 to 20 long acyl chains, from 1 to 10 acyl chains, or from 1 to 6 acyl chains, or 1, 2, 3, 4, 5, 6, 7, 8, 9 or 10 acyl chains. The lipid moieties can be prepared from fatty acids, which include, but are not limited to, capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26).

Exemplary acyl groups in the lipid moieties include C₁₀₋₂₀ acyl chains, such as C₁₀, C₁₂, C₁₄, C₁₆, C₁₈, or C₂₀ acyl groups. In some embodiments, the lipid moieties have at least one C₁₄ acyl group, or at least one C₁₆ acyl group. When the lipid moieties include more than one acyl group, the lipid moiety also includes a branched linker providing for attachment of multiple acyl groups. The branched linkers useful in the present invention include, but are not limited to, lysine, glutamic acid and other branched amines and carboxylic acids. In some embodiments, the lipid moiety includes from 1 to 6 C₁₀₋₂₀ acyl groups. The lipid moiety can include 1, 2, 3, 4, 5 or 6 C₁₀₋₂₀ acyl groups. In some embodiments, the lipid moiety includes 1, 2, or 4 C₁₀₋₂₀ acyl groups. In some embodiments, the lipid moiety includes 1 C₁₀₋₂₀ acyl group. In some embodiments, the lipid moiety includes 2 C₁₀₋₂₀ acyl groups.

In some embodiments, the invention provides a conjugate as described above, wherein the peptide is SEQ ID NO:1, the first polymer is polyethylene glycol with a molecular weight of about 2000 Da, the metal binding ligand comprises 6-aminobenzyl TETA and is linked to the C-terminal residue of the peptide, and the hydrophobic moiety is a lipid moiety which includes lysine and two C₁₈ acyl chains.

The present invention also provides helix bundles, formed from the self-assembly of a plurality of conjugates. The helix bundles can be formed from 2, 3, 4, 5, 6, 7, 8, 9 or 10 conjugates. In some embodiments, the present invention provides a helix bundle having from 2 to 6 conjugates of the present invention. In some embodiments, the helix bundles includes 3 conjugates. In some embodiments, the helix bundle includes 4 conjugates.

The present invention also provides particles formed from the self-assembly of the helix bundles, such that the hydrophobic moiety forms a micellar structure having a hydrophobic core, and helix bundle headgroups are on the exterior of the core. The particles can include any suitable number of conjugates. In some embodiments, the present invention provides a particle having from about 20 to about 200 conjugates of the present invention. In other embodiments, the particles of the present invention can be a mixture of the conjugates of the present invention and those described in U.S. application Ser. No. 14/490,336, incorporated by reference herein in its entirety. The conjugates of U.S. application Ser. No. 14/490,336 are characterized by having a second polymer, instead of the metal binding ligand, covalently linked to the C-terminal amino acid residue of the peptide. Any suitable ratio of the two conjugates can be used in the particles of the present invention.

The particles of the present invention can be of any suitable size. For example, the particles can be from about 5 nm to about 500 nm in diameter, or from about 5 to about 100 nm in diameter, or from about 5 nm to about 50 nm in diameter, or from about 5 nm to about 25 nm in diameter.

The particles of the present invention can include cargo in the hydrophobic interior of the particle. In some embodiments, the particles include at least one additional agent selected from a therapeutic agent, a diagnostic agent, DNA, an oligonucleotide, or other useful agents. Examples of therapeutic agents include, but are not limited to, anthracyclines (such as doxorubicin, daunorubicin, epirubicin, and the like), macrolides (such as rapamycin, fujimycin, pimecrolimus, and the like), alkylating agents (such as temozolomide, procarbazine, altretamine, and the like), taxanes, and vinca alkaloids. Examples of diagnostic agents include, but are not limited to, chromophores, fluorophores, and radionuclides. The conjugates, helix bundles and particles of the present invention can be linked to other particles, such as gold nanoparticles and magnetic nanoparticles that are typically a few nanometers in diameter for imaging and manipulation purposes. In some embodiments, the invention provides particles as described above, wherein each additional agent is independently selected from a fluorophore, a radionuclide, an anthracycline, a taxane, and a macrolide. In some embodiments, each additional agent is independently selected from doxorubicin, paclitaxel, and rapamycin. In some embodiments, the additional agent can be doxorubicin. Alternatively, the additional agents be covalently or noncovalently bound to one of, a combination of, or all of the peptide component, and the first polymeric component of the amphiphilic conjugates.

In some embodiments, the present invention provides a particle having from about 20 to about 200 conjugates of the present invention. Some conjugates include a first peptide having SEQ ID NO:1, a first polymer including polyethylene glycol with a molecular weight of about 2000 Da, a metal binding ligand covalently linked to the C-terminal residue of the peptide and including polyethylene glycol with a molecular weight of about 750 Da, and a hydrophobic moiety having a lipid moiety which includes lysine and two C₁₈ acyl chains. Other conjugates of the particle include a first peptide having SEQ ID NO:1, a first polymer including polyethylene glycol with a molecular weight of about 2000 Da, a second polymer including polyethylene glycol with a molecular weight of about 750 Da covalently linked to the C-terminal residue of the peptide, and a hydrophobic moiety having a lipid moiety which includes lysine and two C₁₈ acyl chains. The particle also includes a therapeutic agent selected from doxorubicin, paclitaxel, and rapamycin. In some embodiments, the therapeutic agent can be doxorubicin.

Additional materials can be incorporated into the particles to form mixed micelles. For example, mixed micelles can include suitable lipid compounds. Suitable lipids can include but are not limited to fats, waxes, sterols, cholesterol, fat-soluble vitamins, monoglycerides, diglycerides, phospholipids, sphingolipids, glycolipids, derivatized lipids, and the like. In some embodiments, suitable lipids can include amphipathic, neutral, non-cationic, anionic, cationic, or hydrophobic lipids. In certain embodiments, lipids can include those typically present in cellular membranes, such as phospholipids and/or sphingolipids. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Non-cationic lipids include but are not limited to dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), di stearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin.

The lipids can also include derivatized lipids, such as PEGylated lipids. PEGylated lipids generally contain a lipid moiety as described herein that is covalently conjugated to one or more PEG chains. The PEG can be linear or branched, wherein branched PEG molecules can have additional PEG molecules emanating from a central core and/or multiple PEG molecules can be grafted to the polymer backbone. PEG can include low or high molecular weight PEG, e.g., PEG500, PEG2000, PEG3400, PEG5000, PEG6000, PEG9000, PEG10000, PEG20000, or PEG50000 wherein the number, e.g., 500, indicates the average molecular weight. Derivatized lipids can include, for example, DSPE-PEG2000, cholesterol-PEG2000, DSPE-polyglycerol, or other derivatives generally well known in the art.

Accordingly, some embodiments of the present invention provide particles as described above further comprising a PEGylated lipid. In some embodiments, the PEGylated lipid can be DSPE-PEG2000. Any suitable amount of PEGylated lipid can be used to form the mixed micelles. In general, the ratio of the PEGylated lipid to the peptide conjugate is from about 0.1:1 to about 10:1 by weight. The ratio of the PEGylated lipid to the helix-bundle conjugate can be, for example, about 0.1:1, 0.5:1, 1:1, 2.5:1, 5:1, or 10:1 by weight. Other amounts of the PEGylated lipid can be useful in the particles of the invention, depending on the structure of the PEGylated lipid itself as well as the identity of the peptide conjugate. In some embodiments, the particles can include DSPE-PEG2000 and a peptide conjugate as described above in a ratio of about 1:1 by weight.

### IV. Methods of Preparing Nanoparticles

The nanoparticles of the present invention can be prepared by any suitable method known to one of skill in the art. For example, the nanoparticles can be prepared by first dissolving the conjugates in a suitable solvent at any concentration from about 1 nM to about 1M, or from about 1 μM to about 100 mM, or from about 1 mM to about 100 mM. Alternatively, the conjugates can be dissolved at a concentration of from about 0.1 to about 50 wt. % of the solution, or from about 1 to about 50 wt. %, or from about 1 to about 25 wt. %. The conjugates self-assemble to form the helix bundles of the present invention. The helix bundles then self-assemble to form the particles. In some embodiments, the present invention provides a method of forming particles of the present invention by maintaining a plurality of conjugates of the present invention under conditions sufficient to allow the conjugates to self-assemble into the particles of the present invention. In some embodiments, the conjugates are at a concentration of from about 1 nM to about 1 M. In some embodiments, the conjugates are at a concentration of from about 1 μM to about 1 M. In some embodiments, the conjugates are at a concentration of from about 1 μM to about 1 100 mM. In some embodiments, the conjugates are at a concentration of from about 1 μM to about 1 mM.

The methods of the invention can also be used to form mixed micelles above. Accordingly, additional compounds such as PEGylated lipids can be used for co-assembly with the peptide conjugates. In some embodiments, the present invention provides a method of forming particles by maintaining a plurality of conjugates under conditions sufficient to allow the conjugates to self-assemble into the particles, and by further adding a PEGylated lipid to the plurality of conjugates.

In an aqueous solvent, the conjugates of the present invention can self-assemble such that the hydrophilic portion is oriented towards the exterior of the nanocarrier and the hydrophobic portion is oriented towards the interior, thus forming a micelle. When a non-polar solvent is used, an inverse micelle can be formed where the hydrophilic portion is oriented towards the interior of the nanocarrier and the hydrophobic portion is oriented towards the exterior of the nanocarrier.

### V. Methods for Drug Delivery

In some embodiments, the present invention provides a method for delivering a diagnostic or therapeutic agent to a subject comprising administering a particle to the subject. In some embodiments, the particle encapsulates the diagnostic or therapeutic agent. In other embodiments, the diagnostic or therapeutic agent is conjugated or coupled to the particle of the present invention. Thus, the particle includes from about 20 to about 200 conjugates of the present invention and the diagnostic or therapeutic agent to be delivered. In some embodiments, the therapeutic agent is selected from the group consisting of doxorubicin, temzolomide, and rapamycin.

Delivery of the therapeutic agent can be conducted such that drug-loaded micelles selectively accumulate at a desired site in a subject, such as a specific organ or a tumor. In some cases, micelle accumulation at a target site may be due to the enhanced permeability and retention characteristics of certain tissues such as cancer tissues. Accumulation in such a manner can arise, in part, from the micelle size and may not require special targeting functionality. In other cases, the micelles of the present invention can also include ligands for active targeting as described above. Target delivery can also be accomplished by administering drug-loaded micelles directed to a desired site. In some embodiments, delivery of a therapeutic agent can include administering a particle of the present invention via intra-tumoral infusion.

The nanoparticles of the present invention can be used to deliver any suitable cargo in a targeted or untargeted fashion. Suitable cargo includes, but is not limited to, vaccines, nucleic acids such as DNA or RNA, peptides, proteins, imaging agents, and drugs. The nanoparticles of the present invention are also useful for gene therapy, the administration of an expressed or expressible nucleic acid to a subject.

The nanocarrier cargo can be encapsulated within the nanocarrier.

### Targeting Agents

Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In certain embodiments, a targeting agent can include a target ligand, a small molecule mimic of a target ligand, or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

### Therapeutic Agents

The therapeutic agent or agents used in the present invention can include any agent directed to treat a condition in a subject. In general, any therapeutic agent known in the art can be used, including without limitation agents listed in the United States Pharmacopeia (U.S.P.), Goodman and Gilman's The Pharmacological Basis of Therapeutics, 10^{th} Ed., McGraw Hill, 2001; Katzung, Ed., Basic and Clinical Pharmacology, McGraw-Hill/Appleton & Lange, 8^{th} ed., Sep. 21, 2000; Physician's Desk Reference (Thomson Publishing; and/or The Merck Manual of Diagnosis and Therapy, 18^{th} ed., 2006, Beers and Berkow, Eds., Merck Publishing Group; or, in the case of animals, The Merck Veterinary Manual, 9^{th} ed., Kahn Ed., Merck Publishing Group, 2005; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, siRNA and/or shRNA agents.

Therapeutic agents can include an anticancer agent or cytotoxic agent including but not limited to avastin, doxorubicin, temzolomide, rapamycin, platins such as cisplatin, oxaliplatin and carboplatin, cytidines, azacytidines, 5-fluorouracil (5-FU), gemcitabine, capecitabine, camptothecin, bleomycin, daunorubicin, vincristine, topotecan or taxanes, such as paclitaxel and docetaxel.

Therapeutic agents of the present invention can also include radionuclides for use in therapeutic applications. For example, emitters of Auger electrons, such as ¹¹¹In, can be combined with a chelate, such as diethylenetriaminepentaacetic acid (DTPA) or 1,4,7,10-tetraazacyclododecane-1,4,7,10-tetraacetic acid (DOTA), and included in a nanoparticle to be used for treatment. Other suitable radionuclide and/or radionuclide-chelate combinations can include but are not limited to beta radionuclides (¹⁷⁷Lu, ¹⁵³Sm, ^{88/90}Y) with DOTA, ⁶⁴Cu-TETA, ^{188/186}Re(CO)₃-IDA; ^{188/186}Re(CO)triamines (cyclic or linear), ^{188/186}Re(CO)₃-Enpy2, and ^{188/186}Re(CO)₃-DTPA.

Diagnostic Agents

A diagnostic agent used in the present invention can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, a diagnostic agent can include chelators that bind to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cb, or ^{67/68}Ga. In some embodiments, the micelles can be radiolabeled, for example, by incorporation of chelating groups, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook-A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to gadopentetic acid, gadoteric acid, gadodiamide, gadolinium, gadoteridol, mangafodipir, gadoversetamide, ferric ammonium citrate, gadobenic acid, gadobutrol, or gadoxetic acid. Superparamagnetic agents can include but are not limited to superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan and iosimenol.

### Gene Therapy

The nanoparticles of the present invention can also be used to deliver any expressed or expressible nucleic acid sequence to a cell for gene therapy or nucleic acid vaccination. The cells can be in vivo or in vitro during delivery. The nucleic acids can be any suitable nucleic acid, such as deoxyribonucleic acid (DNA) or ribonucleic acid (RNA). Moreover, any suitable cell can be used for delivery of the nucleic acids.

Gene therapy can be used to treat a variety of diseases, such as those caused by a single-gene defect or multiple-gene defects, by supplementing or altering genes within the host cell, thus treating the disease. Typically, gene therapy involves replacing a mutated gene, but can also include correcting a gene mutation or providing DNA encoding for a therapeutic protein. Gene therapy also includes delivery of a nucleic acid that binds to a particular messenger RNA (mRNA) produced by the mutant gene, effectively inactivating the mutant gene, also known as antisense therapy. Representative diseases that can be treated via gene and antisense therapy include, but are not limited to, cystic fibrosis, hemophilia, muscular dystrophy, sickle cell anemia, cancer, diabetes, amyotrophic lateral sclerosis (ALS), inflammatory diseases such as asthma and arthritis, and color blindness.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

### Formulation and Administration

When the nanocarriers are administered to deliver the cargo as described above, the nanocarriers can be in any suitable composition with any suitable carrier, i.e., a physiologically acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline, water, buffered water, saline, glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

Prior to administration, the nanocarrier compositions can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized compositions.

The nanocarrier compositions can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged composition with a suppository base. Suitable suppository bases include natural or synthetic triglycerides or paraffin hydrocarbons. In addition, it is also possible to use gelatin rectal capsules which contain a combination of the composition of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration—including intravenous administration—is the preferred method of administration.

Conjugates, particles, and formulations of the present invention can also be delivered by infusion directly into an area of the brain (such as the striatum or a brain tumor) by convection-enhanced delivery (CED), a technique that uses a pressure gradient established at the tip of an infusion catheter to initiate bulk flow that forces the infusate through the space between brain cells (i.e. the extracellular space). An infusion pump or an osmotic pump can be used for CED. Using CED devices, the conjugates, particles, and compositions of the invention can be delivered to many cells over large areas of the brain. CED is described, for example, in U.S. Pat. Nos. 6,953,575; 7,534,613; and 8,309,355.

The pharmaceutical preparation is preferably in unit dosage form. In such form the preparation is subdivided into unit doses containing appropriate quantities of the active component, e.g., a nanocarrier composition. The unit dosage form can be a packaged preparation, the package containing discrete quantities of preparation. The formulations of nanocarrier compositions can be presented in unit-dose or multi-dose sealed containers, such as ampoules and vials. The composition can, if desired, also contain other compatible therapeutic agents.

In therapeutic use, the nanocarrier compositions including a therapeutic and/or diagnostic agent, as described above, can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, can be varied depending upon the requirements of the patient, the severity of the condition being treated, and the nanocarrier composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular nanocarrier composition in a particular patient. Determination of the proper dosage for a particular situation is within the skill of the practitioner. Generally, treatment is initiated with smaller dosages which are less than the optimum dose of the nanocarrier composition. Thereafter, the dosage is increased by small increments until the optimum effect under circumstances is reached. For convenience, the total daily dosage can be divided and administered in portions during the day, if desired.

### Loading of Nanocarriers

Loading of the diagnostic and therapeutic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006). In some embodiments, one or more therapeutic agents can be loaded into the nanocarriers. Loading of nanocarriers can be carried out, for example, in an active or passive manner. For example, a therapeutic agent can be included during the self-assembly process of the nanocarriers in a solution, such that the therapeutic agent is encapsulated within the nanocarrier. In certain embodiments, the therapeutic agent may also be embedded in the lamellar layer. In alternative embodiments, the therapeutic agent can be actively loaded into the nanocarriers. For example, the nanocarriers can be exposed to conditions, such as electroporation, in which the lamellar membrane is made permeable to a solution containing therapeutic agent thereby allowing for the therapeutic agent to enter into the internal volume of the liposomes.

The diagnostic and therapeutic agents can also be covalently or ionically linked to the surface of the nanocarrier, in the interior of the micelle, or within the lamellar layer of the micelle.

### VI. Methods for Disease Treatment

In some embodiments, the present invention provides a method for treating a subject with a disease. The method includes administering a therapeutically effective amount of a particle to the subject. The particle includes from about 20 to about 200 conjugates of the present invention and at least one therapeutic agent. Thus, the disease is treated.

Any suitable disease can be treated using the conjugates and particles of the present invention. Representative diseases include cancer and Parkinson's disease, among others. Cancers contemplated for treatment using the methods of the present invention include carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, brain and central nervous system, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, Hodgkin's lymphoma, and non-Hodgkin's lymphoma. In some embodiments, the present invention provides a method for treating a subject with a cancer characterized by solid tumors. In some embodiments, the disease is selected from the group consisting of a cancer and Parkinson's disease. In some embodiments, the cancer is Glioblastoma multiforme.

In some embodiments, the present invention provides a method for treating a subject with brain cancer. Brain cancers include gliomas, meningiomas, pituitary adenomas, and nerve sheath tumors. In some embodiments, the brain cancer is Glioblastoma multiforme. Glioblastoma multiforme presents variants including giant cell glioblastoma and gliosarcoma.

The particles of the invention can be used in conjunction or concurrently with other known methods of disease treatment, including—but not limited to—chemotherapy and radiotherapy. Any suitable therapeutic agent is useful in combination with the conjugates and particles of the present invention. In some embodiments, the therapeutic agent is selected from the group consisting of doxorubicin, temzolomide, and rapamycin. In some embodiments, the at least one therapeutic agent can be doxorubicin, paclitaxel or rapamycin. In other embodiments, the therapeutic agent is doxorubicin.

In some embodiments, the present invention provides a method of treating a disease state in a human subject, including administering a therapeutically effective amount of a particle of the present invention, and at least one therapeutic agent. In some embodiments, the disease state can be cancer, autoimmune disorders, genetic disorders, infections, inflammation, neurologic disorders, or metabolic disorders. In some embodiments, the therapeutic agent can be a vaccine, nucleic acid or peptide.

### VII. Examples

### Materials and Methods

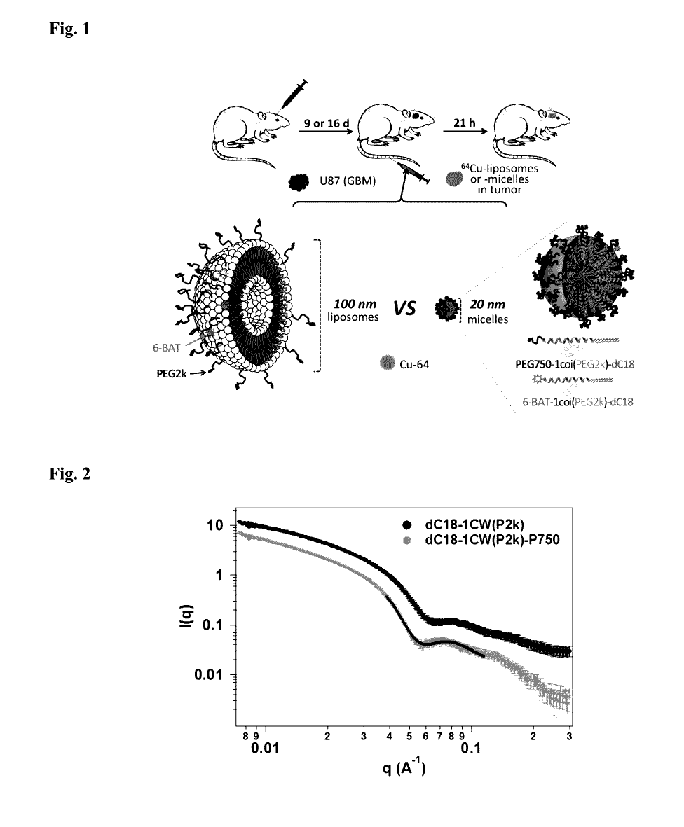

An overview of the experimental procedures is provided in FIG. 1. HSPC, cholesterol, DSPE-PEG2k-OMe, were purchased from Avanti Polar Lipids (Alabaster, Ala.), Solvents and other agents were all of analytical purity and purchased from Sigma-Aldrich (Milwaukee, Wis.) and VWR (Brisbane, Calif.). ⁶⁴CuCl₂ was purchased from MIR Radiological Science (St. Louis, Mo.) under a protocol controlled by the University of California Davis. Phosphate-buffered saline (PBS) was purchased from Invitrogen Corporation (Carlsbad, Calif.).

The 3HM amphiphile can be readily synthesized using solid phase peptide synthesizer. Detailed chemistry and purification procedure have been documented in detail [40]. Briefly, the amphiphile is based on a 3-helix bundle forming peptide, 1COI (EVEALEKKVAALECKVQALEKKVEALEHGW)[45, 46]. The peptide was synthesized on a Prelude solid phase peptide synthesizer (Protein Technologies) using standard 9-fluorenylmethyl carbamate (Fmoc) chemistry. For the synthesis of amphiphilic subunits, the alkyl chains were conjugated through reaction of stearic acid (C18) with deprotected Fmoc-Lys(Fmoc)-OH to generate a branched alkyl tail at the N-terminus. Modification of the C-terminus was achieved through orthogonal protection strategy employed in Fmoc-SPPS. The resulting free amino groups of lysine were utilized for conjugating carboxy-terminated fluorescein using HBTU/DIPEA chemistry. Cleavage was carried out using a cocktail of 90:8:2 TFA/TIS/water for 3 h. Crude peptides were precipitated in cold ether, isolated, and dried for the conjugation of polymers. To conjugate PEG, cysteine at position 14 facilitates the site-specific coupling of maleimide-functionalized PEG of molecular weight 2000 g/mol to the middle of the peptide sequence. The conjugation reaction was carried out in phosphate buffer (pH=6.2) overnight with a reaction ratio of PEG to peptide at 5:1. Cysteine at the C-terminus of 1coi-dC18-PEG2K allows for the conjugation of either PEG(750 Da) or 6-BAT-maleimide onto the peptides for PET imaging.

### 3HM and Characterization

After dissolving the lyophilized amphiphile powder into aqueous solution, dynamic light scattering (DLS) reveals a hydrodynamic diameter of ˜20 nm and a fairly uniform size distribution of micelles. We further performed solution small angle x-ray scattering studies to verify the particle size and the outer PEG layer thickness. The surface property of the micelle has significant effects on the in vivo behavior of nanocarrier. Although previous in vivo studies confirmed the effective stealth PEG layer on the 3HM surface, it is important to determine the PEG 750 conformation and the PEG brush layer density.

### Small-Angle X-Ray Scattering of 3HM

Small-angle x-ray scattering (SAXS) experiments were carried out at the Advanced Light Source (ALS) at the Lawrence Berkeley National Lab, Berkeley, Calif. at the SAXS/WAXS/GISAXS beamline 7.3.3. The instrument was operated using an X-ray energy of 10 keV and a sample—detector length of 1.2 m and a 1 M Pilatus detector. Samples were contained in standard boron-quartz capillaries situated in a homemade sample holder. Using this setup, background subtraction could be made quantitatively. Samples were dissolved in phosphate buffer (25 mM, pH 7.4) at a concentration of ˜5 mg/ml, annealed at 70° C. for 1 hour and allowed to equilibrate at room temperature overnight before SAXS measurements were performed.

### Animal Model

All animal experiments were conducted under a protocol approved by the University of California, Davis, Animal Use and Care Committee (Davis, Calif.). Eleven male athymic nude rats were purchased from Harlan Laboratories (Hayward, Calif.) and weighed ˜250 g upon arrival. U87MG cells at 3×10⁶ cells/10 μL were intracranially inoculated through a small burr hole in the skull into the right striatum of each rat. Imaging studies were completed at nine (n=6) and sixteen days (n=5) post-surgery; at this time the age ranged from 82 to 93 days and the average body weight was 294±35 g.

### Positron Emission Tomography/Magnetic Resonance (PET/MR) Imaging

Radioactivity was handled under a university-approved radiation use authorization (Davis, Calif.). Glioblastoma-bearing rats were administered 200 μL of ⁶⁴Cu-liposomes (690±325 μCi, 4.15±0.75 mg, n=6) and ⁶⁴Cu-micelles (284±97 μCi, 4.22±0.99 mg, n=5) via tail vein under 1.5% isoflurane anesthesia. The critical micelle concentration (CMC) tested in this experiment is ˜4 μM (˜0.03 mg/mL). Thus, the micelle concentration (0.23 mg/mL) (calculated by dividing the average dose (4.22±0.99 mg) of micelles by estimated blood volume (18.4 mL) [47]) was seven times higher than the CMC.

PET images were acquired with a Focus 120 scanner (Siemens Medical Solutions Inc., Malvern, Pa.) over 30 minutes at 0, 3.5, 7, and 21 h after injection of nanoparticles. After PET scanning at 21 hours, MR imaging was immediately performed with a Bruker Biospec 7 Tesla (7T) small-animal scanner (Bruker BioSpin MRI, Ettlingen, Germany). A 72-mm internal diameter linear resonator was used for RF transmission and a four-channel rat brain phased array surface coil was used for signal reception. Rat brains were imaged coronally with a fast-spin echo sequence (“RARE”; axial: TE/TR=8 ms/750 ms; FOV=40×40 mm²; MTX=256×256; ST/SI=1 mm/1 mm; ETL=4. Coronal: TE/TR=9 ms/1200 ms; FOV=50×30 mm²; MTX=320×192; ST/SI=1 mm/1 mm; ETL=4.). Data were acquired and reconstructed with ParaVision 5.1 software (Bruker BioSpin MM). PET/MR images were co-registered on Inveon Research Workspace 4.2 (Siemens Medical Solutions Inc., USA)

### Biodistribution

After PET/MR imaging, animals were immediately euthanized with Euthasol (Western Medical Supply, Arcadia, Calif.). Blood was collected by syringe from the left ventricle and perfused from the body with Dulbecco's Modified Eagle's medium (DMEM, Invitrogen, Carlsbad, Calif.). Heart, lungs, stomach, intestine, muscle, bone, liver, kidneys, spleen and brain were harvested and placed in a gamma counter (Perkin-Elmer life Sciences). Values are presented as % injected dose per gram (% ID/g).

### Image Analysis for Pharmacokinetics

All PET images were reconstructed with the maximum a posteriori (MAP) reconstruction algorithm and analyzed with AsiPro software (Onccorde Microsystems Inc., Knoxville, Tenn.) and Inveon Research Workspace 4.2 (Siemens Medical Solutions Inc., USA). ROIs within the glioblastoma and contralateral left brain (striatum) were drawn on co-registered PET/MR images with a volume ranging from 38 to 201 mm³ and 33 to 138 mm³, respectively. Regions of interest (ROIs) in the contralateral brain (striatum) were of a similar size and location to those applied in the tumor. The radioactivity within the blood pool was obtained using ROIs in the heart chamber from the PET images.

Time-activity curves (TAC) of blood radioactivity subtracted ⁶⁴Cu-liposomes and ⁶⁴Cu-micelles uptake in glioblastoma were obtained by equation 1 and values are given in Table S2,

R_{GBM,real}(t)=R_{GBM,ROI}(t)−% VB_{GBM}(t₀)×R_{Blood,ROI}(t) Eq. 1,

where R_{GBm,ROI}(t) is the tumor radioactivity (% ID/cc) at any given time point (t), % VB_{GBM}(t₀) is the percent blood volume in glioblastoma measured at the 0 h time point (t₀) and R_{Blood,ROI}(t) is the radioactivity (% ID/cc) of blood at each time point.

The percent tumor blood volume (TBV) and left brain blood volume (LBV) was calculated by equation 2. TBV and LBV are presented as percent vascular volume in glioblastoma and left brain (striatum)

$$\% Blood volume = \frac{{R}_{GBM , ROI} ( {t}_{0} ) or {R}_{LB , ROI} ( {t}_{0} )}{{R}_{blood , ROI} ( {t}_{0} )} , \qquad Eq . 2$$

where R_{GBM,ROI}) (t₀) and R_{LB,ROI}(t₀) are the radioactivity (% ID/cc) in the glioblastoma and left brain (striatum) at the 0 h time point (t₀), R_{Blood,ROI}(t₀) is the radioactivity (% ID/cc) of blood at the 0 h time point (t₀).

To determine the circulation half-life of the ⁶⁴Cu-liposomes and ⁶⁴Cu-micelles, the % ID/cc obtained from the ROI image was fitted to a one-phase decay curve using Prism 6 for Mac OS X software (La Jolla, Calif.). Data are presented as percent injected dose per cubic centimeter (% ID/cc).

### Autoradiography

At necropsy, the sample was placed in Tissue-Tek O.C.T. compound (Sakura Finetek, Torrance, Calif.), frozen down in a mixture of isopropanol and dry ice and placed in the cryostat (Leica Microsystems Inc, Buffalo Grove, Ill.) to equalize the temperature. The sample was then mounted on the cutting stage with O.C.T. and 10-20 μm slices were taken in succession from the front of the brain to the rear of the brain. Slices were adhered onto glass slides (Fisher Scientific, Waltham, Mass.) and once dry were placed on an autoradiography cassette and exposed to the Storage Phosphor Screen (Molecular Dynamics, Sunnyvale, Calif.) for 24 h before analysis on a Phosphor Imager STORM 860 (Amersham Biosciences, NJ). Contrast of the autoradiography image was normalized with the injected dose to compare the intensity of particle accumulation within the brain tumor.

### H&E and Immunohistochemistry (IHC)

Tissues for microscopic analysis were fixed overnight in 4% buffered formalin and transferred to 70% ethanol the next day. A Tissue-Tek VIP autoprocessor (Sakura, Torrance, Calif.) was used to process samples for paraffin-embedding. Tissue blocks were then sectioned to 4 μm, sections mounted on glass slides, then stained with Mayer's hematoxylin and eosin. Samples were processed for immunohistochemistry (IHC) with a goat anti-mouse PECAM-1 (CD31) primary antibody (1:1600; SC-1506, Santa Cruz Biotechnology, Santa Cruz, Calif.). All IHC was performed manually. Antigen retrieval was performed in a Decloaking Chamber (Biocare Medical, Concord, Calif.) with citrate buffer at pH 6.0, 125° C. and pressure of 15 psi within 45 min. Incubation with the primary antibody was performed at room temperature overnight in an humidified chamber and normal horse serum was used for blocking. Biotinylated horse anti-goat (1:1000; Vector Labs, Burlingame, Calif.) was the secondary antibody used with a Vectastain ABC Kit Elite and a Peroxidase Substrate Kit DAB (both from Vector Labs), which were used for amplification and visualization of the signal, respectively. Tissues known to contain the assessed antigen were used as positive controls.

### Statistical Methods

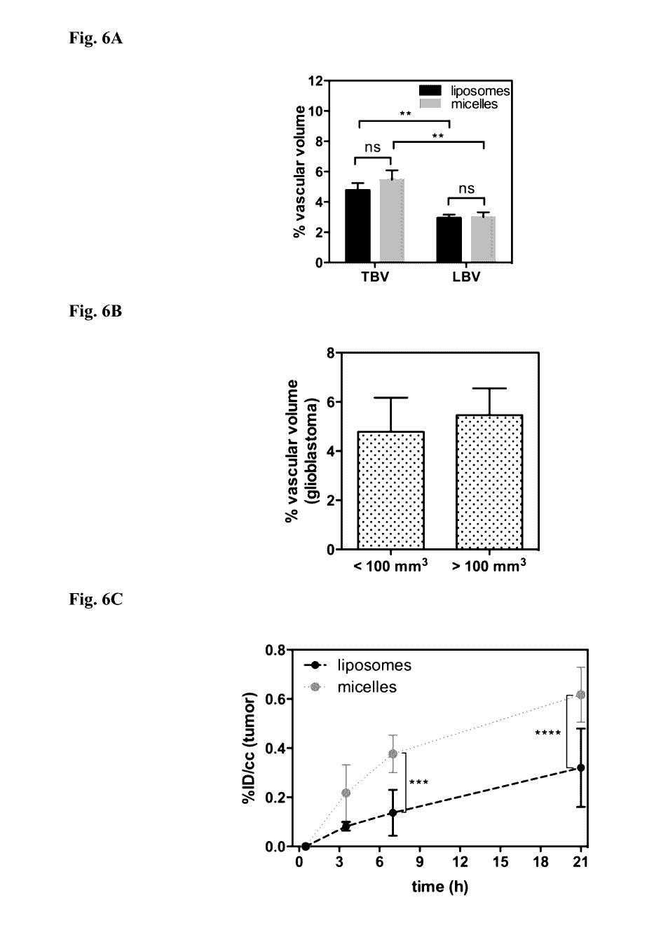

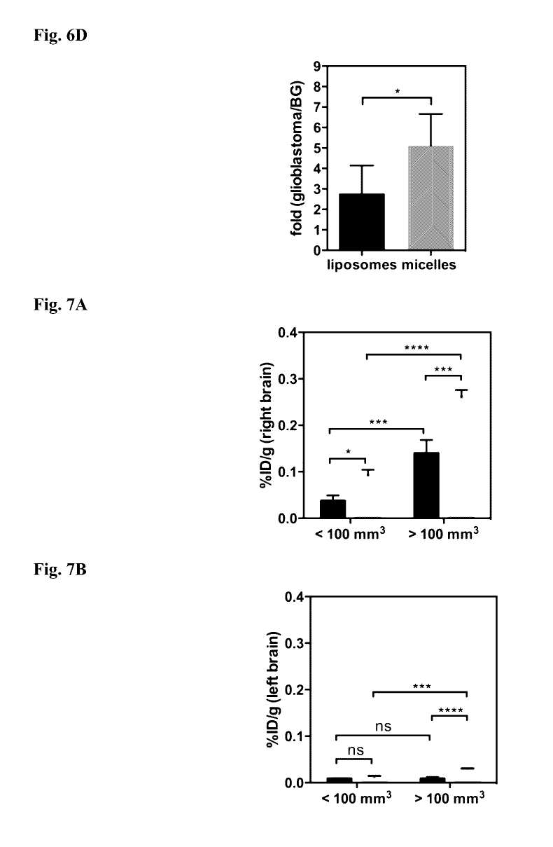

Values are presented as means±S.E.M. Statistical analyses were conducted using GraphPad Prism (v6). For the statistical analysis of tumor accumulation of liposomes and micelles (FIGS. 5a, 5c, 6a and 6b), two-way ANOVA corrected by Sidak's multiple comparisons was performed. Other values were analyzed using unpaired t-test (two-tailed) with Welch's correction. A corrected P value of *<0.05 was considered significant.

### Example 1. Preparation of ⁶⁴Cu-Labeled Liposomes and Micelles

Preparation of liposomes and micelles followed our previously-reported methods [39, 40]. In brief, for liposome preparation: in a glass test tube, the dried lipid film (20 mg, HSPC:6-BAT-lipid:DSPE-PEG2k-OMe:cholesterol=55.5:0.5:5:39, mole percent) was suspended in 0.1 M ammonium citrate buffer (pH 5.5, 0.5 mL) and the solution was incubated for 30 min at 60° C. The lipid mixture was then extruded 21 times through mini extruder with a 100-nm membrane filter under 60-65° C. heating block. After cooling, the solution was kept at room temperature until ⁶⁴Cu labeling was complete. For micelle preparation, dC18-1COI(P2k)-P750 with 2 mol % dC18-1COI(P2k)-6-BAT(10 mg) was dissolved in double-distilled water (0.5 mL) and spontaneously self-assembled into micelles with incubation at 50° C. until the solution became clear (approximately 1 h). Particle size and zeta-potential were measured via dynamic light scattering (DLS) with a Zetasizer Nano (Malvern Instruments Inc., Westborough, Mass.).

Liposomes (0.2 mL of 40 mg/mL solution) and micelles (0.4 mL of a 20 mg/mL solution) were added to ⁶⁴CuCl₂ (Washington University, MA) buffered in 0.1 M ammonium citrate (pH 5.5, 0.1 mL) and incubated for 50 minutes. 0.1 M EDTA (20 mL) in double-distilled water was added in order to remove the non-specifically bound ⁶⁴Cu from the particles. Completion of ⁶⁴Cu labeling was monitored by instant thin-layer chromatography (ITLC) eluted by a 0.1 M ammonium citrate solution (pH 5.5). The chemical purity of isolated ⁶⁴Cu-liposomes and ⁶⁴Cu-micelles after size-exclusion column chromatography (Sephadex-G75 superfine, 6 mL bed volume, DPBS) was determined by ITLC.

### Example 2. Results

### Preparation of ⁶⁴Cu-Liposomes and -Micelles

To facilitate post-labeling, a custom lipid-PEG-chelator conjugate is incorporated into the self-assembled liposomes and micelles. As illustrated in FIG. 1, liposomes with 0.5 mol % 6-BAT lipid and micelles with 2 mol % of dC18-1COI(P2k)-6-BAT were successfully prepared in 0.1 M ammonium citrate buffer (pH 5.5) and deionized water, respectively. The average mean diameter of the liposomes and micelles was 111.9±5.7 and 19.6±7.4 nm, respectively (Table 1). The Z-average particle size of the liposomes was about 6-fold greater than that of the micelles (FIG. 1). The zeta-potential of the liposomes and micelles was −15.6±3.5 and -13.6±1.4 mV under physiological pH, where the negative charge of micelles and liposomes results from PEG on the surface. ⁶⁴Cu was efficiently incorporated into the 6-BAT chelator on both particles resulting in an 80±19% radiolabeling yield, which is comparable to the previous reports [39, 40]. The radiochemical purities of the liposomes and micelles measured by ITLC were above 98% after size-exclusion chromatography. The specific activities of the liposomes and micelles were 159±50.1 μCi/mg (115.6±36.4 μCi/μmol_{lipid}) and 75.3±40.7 μCi/mg (559.8±303.1 μCi/μmol_{lipid}), respectively. The specific activity of both particles was sufficient to evaluate the pharmacokinetics within the glioblastoma model.

TABLE 1
Characterization of liposomes and micelles with
particle size and Zeta potential

| | Liposomes | Micelles |
|---|---|---|
| Z-average size | 111.9 ± 5.7 | 19.6 ± 7.4 |
| (mean ± SD, nm)^{a} | | |
| Zeta-potential (mean ± | −15.6 ± 3.5 | −13.6 ± 1.4 |
| SD, mV)^{a} | | |

^{a}Average mean and standard deviation is calculated from two means of particles used for two in vivo experiments under physiological pH (7.3-7.5).

### Physiochemical Characterization of the 3HM

3HM has been thoroughly characterized using TEM, DLS as reported previously[40]. To extract PEG shell thickness on the outer layer of 3HM, solution SAXS experiments were performed. FIG. 2 shows the solution SAXS profiles of 3HM with and without PEG750 attached to the micelle surface. A core-shell form factor model was used to fit the SAXS data and the parameters of best fit are listed in Table 2. Based on these data, the PEG750 chains form an outer layer with a thickness of ˜0.8 nm.

TABLE 2
Core-shell parameters from model fitting of SAXS data in FIG. 2.

| | Core radius (nm) | Shell thickness (nm) |
|---|---|---|
| w/o P750 | 3.0 | 5.2 |
| with P750 | 3.0 | 6.0 |

### In Vivo PET/MR Imaging

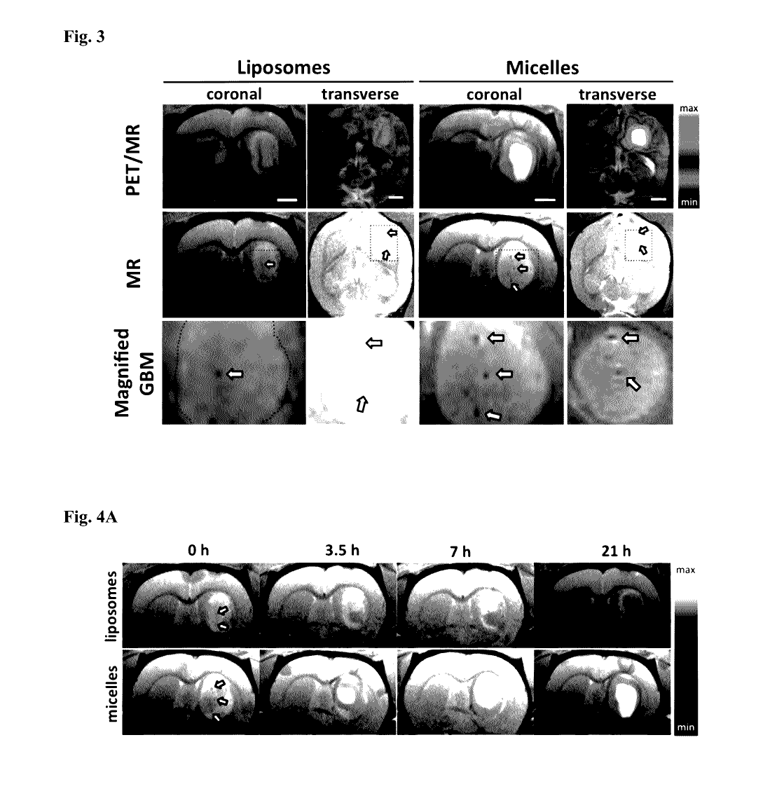

T1w MM contrast (without injection of an exogenous contrast agent) was sufficient to visualize the glioblastoma lesion in the right brain (FIG. 3, lower row) and large blood vessels (white arrows in FIGS. 3 & 4) in the tumor center. MR images in FIG. S1 demonstrated that intracranial injection of U87MG cells in the right brain resulted in a highly localized GBM within the right brain. The average tumor volume at 9 days after surgery (n=6) was <100 mm³ (50±15 mm³) and 16 days after surgery (n=5) was >100 mm³ (154±36 mm³) (FIG. S2).

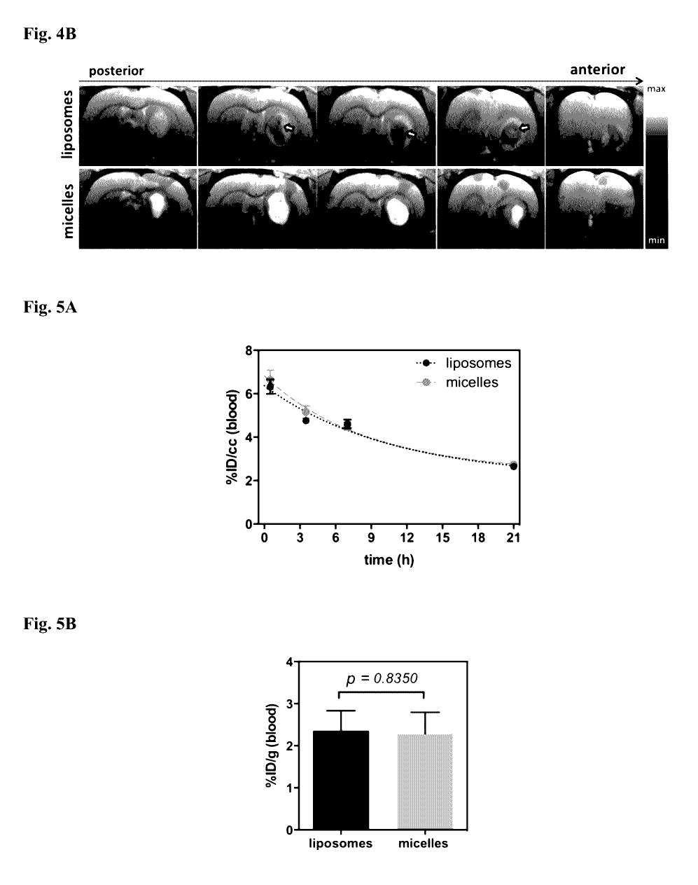

Co-registered PET/MR images obtained 21 h after injection of ⁶⁴Cu-liposomes and -micelles depict the enhanced accumulation of both particles within the tumor as compared with the adjacent striatum in the left brain (FIG. 3). Accumulation of particles increased gradually from 0.5 to 21 h, with evident accumulation of micelles from the 3.5 h time point (FIG. 4a). The accumulation of 20-nm ⁶⁴Cu-micelles was substantially greater than that observed for 110-nm ⁶⁴Cu-liposomes.

Radioactivity associated with both of the ⁶⁴Cu-labeled nanoparticles was first observed in the center of the tumor (3.5 h vs 21 h, FIG. 4a), reaching the periphery at later time points. ⁶⁴Cu-liposomes were also observed to localize around large vessels within the tumors (white arrow, upper row in FIG. 4b). At 21 hours after injection, serial brain slices of the PET/MR images from posterior to anterior (FIG. 4b) also demonstrate that the liposome and micelle concentration remained greater in the tumor center than in the periphery. No significant differences in the relative intratumoral distribution were observed (FIG. S3).

### Pharmacokinetics and Biodistribution of Liposomes and Micelles in Blood

The pharmacokinetics of liposomes and micelles in blood were measured from the ROI analysis of radioactivity in the cardiac chambers. The clearance of ⁶⁴Cu-liposomes and ⁶⁴Cu-micelles in the blood pool was fit by a one-phase decay curve. The half-clearance time of liposomes and micelles was 16.5 and 15.5 h, respectively (FIG. 5a). Radioactivity quantified for ⁶⁴Cu-liposomes (2.36±0.47% ID/g, n=6) and -micelles (2.29±0.50% ID/g, n=5) from blood collected at 22 h after injection (FIG. 5b) was similar to the image-derived values (liposomes: 2.64±0.16% ID/cc, micelles: 2.74±0.35% ID/cc). The slightly lower values calculated for the image-derived estimates are expected due to partial volume effects.

### Calculation of Tumor Blood Volume (TBV) and Left Brain (Striatum) Blood Volume (LBV)

It has been reported that tumor blood volume changes with tumor grade [48, 49]. Indeed, cerebral blood volume (CBV) of C6 gliomas measured by a previous MR study increased by 15% compared to control brain tissue [50]. Here, we segmented the study into liposomes and micelles. At early time points after intravenous administration, long-circulating nanoparticles were only detected in the blood pool and provided a tool for evaluating the TBV by dividing the tumor radioactivity by the blood radioactivity at the 0 h time point [51].

Within this study, the average TBV and LBV measured by liposomes and micelles were not significantly different (FIG. 6a). However, the tumor blood volume in the contralateral left brain (striatum) was significantly lower than that obtained from the glioblastoma irrespective of particle size (FIG. 6a). Finally, in the two tumor size groups, the percent vascular volume within the glioblastoma was similar (<100 mm³: 4.785±1.385%, >100 mm³: 5.462±1.085%, P=0.3867) as shown in FIG. 6b.

### Image Analysis of Glioblastoma and Contralateral Left Brain

The size-dependent accumulation of the nanoparticles in glioblastoma is summarized in the time-activity curves (% ID/cc) (FIG. 6c). Blood-pool radioactivity within the tumor was subtracted from the total local radioactivity by equation 1. At 30 min after injection of the nanoparticles, the radioactivity of the liposomes (0.082±0.018% ID/cc) and micelles (0.217±0.115% ID/cc) within the tumor was not significantly different. Based on the image data, glioblastoma accumulation of the ⁶⁴Cu-micelles was significantly higher than that of ⁶⁴Cu-liposomes at 7 and 21 h after injection reaching a ratio of 1.9 times greater (FIG. 6c). The contralateral left brain (striatum) was used to estimate the background radioactivity, and the tumor to background ratio of the ⁶⁴Cu-micelles (5.12±1.54 fold) was significantly higher than that of ⁶⁴Cu-liposomes (2.78±1.38, FIG. 6d).

### Biodistribution of Liposomes and Micelles

The biodistribution of the liposomes and micelles was then measured after perfusion of animals with Dulbecco modified eagle medium (DMEM) which was used to eliminate the remaining radioactivity contributed by the circulating nanoparticles (˜2% ID/g). The radioactivity within the glioblastoma-bearing right brain and left brain was gamma-counted without tumor dissection.

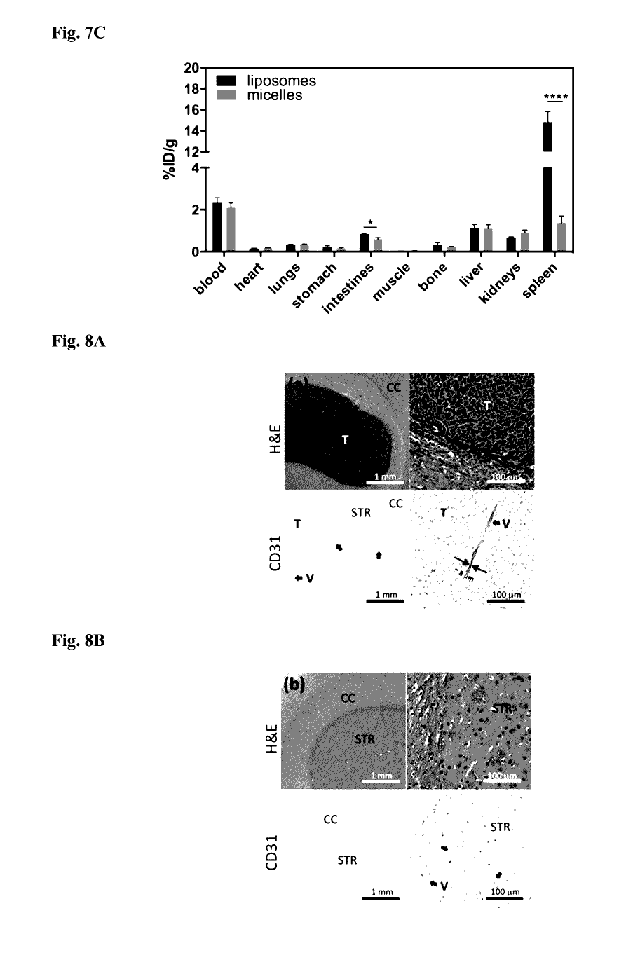

The increased accumulation of micelles within the right brain (containing the glioblastoma), as compared with liposomes, was validated by biodistribution. In FIG. 7a, the accumulation of micelles and liposomes was 0.0924±0.0012% ID/g (n=3) and 0.0372±0.012% ID/g (n=3, p=0.0048), respectively, in the right brain bearing a small tumor. This compares with (0.261±0.015% ID/g, n=3) and (0.140±0.029% ID/g, n=2, p=0.0086) for micelles and liposomes, respectively, in large xenograft. In addition, the accumulation of both liposomes (p=0.0143) and micelles (p=0.0075) was greater in larger xenografts relative to smaller.

Surprisingly, in the contralateral left brain, accumulation of the micelles was also increased relative to that of the liposomes and the accumulation further increased in advanced xenograft peaking at 0.0304±0.00041% ID/g (FIG. 7b).

The uptake of both nanoparticles in other organs (heart, lung, stomach, intestines, muscle, bone, liver and kidneys) was similar 22 h after injection; however, splenic uptake of ⁶⁴Cu-micelles (1.39±0.70% ID/g, n=5) was significantly lower in comparison to the ⁶⁴Cu-liposomes (14.8±2.5% ID/g, n=6, p<0.0001, FIG. 7c). Intestinal radioactivity after the injection of liposomes was significantly higher, although the difference was only ˜1% ID/g.

### Autoradiography and Immunohistochemistry

Optical images (upper row, FIG. S5) and autoradiography from the same slides (lower row, FIG. S5) confirmed the finding of enhanced tumor radioactivity within the co-registered PET/MR images (FIG. 4a). Histological examination (FIG. 8a,b) with H&E (upper row) and with CD31 (lower row) depicts scattered large blood vessels apparent within the glioblastoma with greater frequency as compared with the surrounding tissue. No evidence of tumor was observed in the contralateral (left) brain.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

SEQUENCE LISTING

SEQ ID NO: 1
EVEALEKKVAALECKVQALEKKVEALEHGW
SEQ ID NO: 2
GGGEIWKLHEEFLCKFEELLKLHEERLKKM
SEQ ID NO: 3
AYSSGAPPMPPF
SEQ ID NO: 4
EGKAGEKAGAALKCGVQELEKGAEAGEGGW
SEQ ID NO: 5
EVEALEKKVAALESKVQALEKKVEALEHGW
SEQ ID NO: 6
EVEALEKKVAALECKVQALEKKVEALEHGWGGGK

### REFERENCES

[1] D. N. Louis, H. Ohgaki, O. D. Wiestler, W. K. Cavanee, WHO classification of tumours of the central nervous system. 4th ed. Lyon, France: IARC Press, (2007).
[2] M. Preusser, S. de Ribaupierre, A. Wohrer, S. C. Erridge, M. Hegi, M. Weller, R. Stupp, Current Concepts and Management of Glioblastoma, Ann Neurol, 70 (2011) 9-21.
[3] P. Y. Wen, S. Kesari, Malignant gliomas in adults, New Engl J Med, 359 (2008) 492-507.
[4] F. Zustovich, G. Lombardi, A. Della Puppa, A. Rotilio, R. Scienza, D. Pastorelli, A Phase II Study of Cisplatin and Temozolomide in Heavily Pre-treated Patients with Temozolomide-refractory High-grade Malignant Glioma, Anticancer Res, 29 (2009) 4275-4279.
[5] A. A. Brandes, U. Basso, M. Reni, F. Vastola, A. Tosoni, G. Cavallo, L. Scopece, A. J. Ferreri, M. G. Panucci, S. Monfardini, M. Ermani, First-line chemotherapy with cisplatin plus fractionated temozolomide in recurrent glioblastoma multiforme: A phase II study of the Gruppo Italiano Cooperativo di Neuro-Oncologia, J Clin Oncol, 22 (2004) 1598-1604.
[6] G. B. Zhang, S. Y. Huang, Z. C. Wang, A meta-analysis of bevacizumab alone and in combination with irinotecan in the treatment of patients with recurrent glioblastoma multiforme, J Clin Neurosci, 19 (2012) 1636-1640.
[7] J. J. Vredenburgh, A. Desjardins, D. A. Reardon, H. S. Friedman, Experience with irinotecan for the treatment of malignant glioma, Neuro-Oncology, 11 (2009) 80-91.
[8] H. S. Friedman, M. D. Prados, P. Y. Wen, T. Mikkelsen, D. Schiff, L. E. Abrey, W. K. A. Yung, N. Paleologos, M. K. Nicholas, R. Jensen, J. Vredenburgh, J. Huang, M. X. Zheng, T. Cloughesy, Bevacizumab Alone and in Combination With Irinotecan in Recurrent Glioblastoma, J Clin Oncol, 27 (2009) 4733-4740.
[9] G. M. Marx, N. Pavlakis, S. McCowatt, F. M. Boyle, J. A. Levi, D. R. Bell, R. Cook, M. Biggs, N. Little, H. R. Wheeler, Phase II study of Thalidomide in the treatment of recurrent glioblastoma multiforme, J Neuro-Oncol, 54 (2001) 31-38.
[10] V. K. Puduvalli, P. Giglio, M. D. Groves, K. R. Hess, M. R. Gilbert, S. Mahankali, E. F. Jackson, V. A. Levin, C. A. Conrad, S. H. Hsu, H. Colman, J. F. de Groot, M. G. Ritterhouse, S. E. Ictech, W. K. A. Yung, Phase II trial of irinotecan and thalidomide in adults with recurrent glioblastoma multiforme, Neuro-Oncology, 10 (2008) 216-222.
[11] M. R. Gilbert, J. J. Dignam, T. S. Armstrong, J. S. Wefel, D. T. Blumenthal, M. A. Vogelbaum, H. Colman, A. Chakravarti, S. Pugh, M. Won, R. Jeraj, P. D. Brown, K. A. Jaeckle, D. Schiff, V. W. Stieber, D. G. Brachman, M. Werner-Wasik, I. W. Tremont-Lukats, E. P. Sulman, K. D. Aldape, W. J. Curran, M. P. Mehta, A Randomized Trial of Bevacizumab for Newly Diagnosed Glioblastoma, New Engl J Med, 370 (2014) 699-708.
[12] O. L. Chinot, W. Wick, W. Mason, R. Henriksson, F. Saran, R. Nishikawa, A. F. Carpentier, K. Hoang-Xuan, P. Kavan, D. Cernea, A. A. Brandes, M. Hilton, L. Abrey, T. Cloughesy, Bevacizumab plus Radiotherapy-Temozolomide for Newly Diagnosed Glioblastoma, New Engl J Med, 370 (2014) 709-722.
[13] R. Stupp, M. E. Hegi, W. P. Mason, M. J. van den Bent, M. J. B. Taphoorn, R. C. Janzer, S. K. Ludwin, A. Allgeier, B. Fisher, K. Belanger, P. Hau, A. A. Brandes, J. Gijtenbeek, C. Marosi, C. J. Vecht, K. Mokhtari, P. Wesseling, S. Villa, E. Eisenhauer, T. Gorlia, M. Weller, D. Lacombe, J. G. Cairncross, R. O. Mirimanoff, E. O. R. Treatment, C. B. T. Grp, R. O. Grp, N.C.I.C.C. Trials, Effects of radiotherapy with concomitant and adjuvant temozolomide versus radiotherapy alone on survival in glioblastoma in a randomised phase III study: 5-year analysis of the EORTC-NCIC trial, Lancet Oncol, 10 (2009) 459-466.
[14] R. Stupp, W. P. Mason, M. J. van den Bent, M. Weller, B. Fisher, M. J. B. Taphoorn, K. Belanger, A. A. Brandes, C. Marosi, U. Bogdahn, J. Curschmann, R. C. Janzer, S. K. Ludwin, T. Gorlia, A. Allgeier, D. Lacombe, J. G. Cairncross, E. Eisenhauer, R. O. Mirimanoff, D. Van Den Weyngaert, S. Kaendler, P. Krauseneck, N. Vinolas, S. Villa, R. E. Wurm, M. H. B. Maillot, F. Spagnolli, G. Kantor, J. P. Malhaire, L. Renard, O. De Witte, L. Scandolaro, C. J. Vecht, P. Maingon, J. Lutterbach, A. Kobierska, M. Bolla, R. Souchon, C. Mitine, T. Tzuk-Shina, A. Kuten, G. Haferkamp, J. de Greve, F. Priou, J. Menten, I. Rutten, P. Clavere, A. Malmstrom, B. Jancar, E. Newlands, K. Pigott, A. Twijnstra, O. Chinot, M. Reni, A. Boiardi, M. Fabbro, M. Campone, J. Bozzino, M. Frenay, J. Gijtenbeek, A. A. Brandes, J. Y. Delattre, U. Bogdahn, U. De Paula, M. J. van den Bent, C. Hanzen, G. Pavanato, S. Schraub, R. Pfeffer, R. Soffietti, M. Weller, R. D. Kortmann, M. Taphoorn, J. L. Torrecilla, C. Marosi, W. Grisold, P. Huget, P. Forsyth, D. Fulton, S. Kirby, R. Wong, D. Fenton, B. Fisher, G. Cairncross, P. Whitlock, K. Belanger, S. Burdette-Radoux, S. Gertler, S. Saunders, K. Laing, J. Siddiqui, L. A. Martin, S. Gulavita, J. Perry, W. Mason, B. Thiessen, H. Pai, Z. Y. Alam, D. Eisenstat, W. Mingrone, S. Hofer, G. Pesce, J. Curschmann, P. Y. Dietrich, R. Stupp, R. O. Mirimanoff, P. Thum, B. Baumert, G. Ryan, E.O.R.T.C. Br, Radiotherapy plus concomitant and adjuvant temozolomide for glioblastoma, New Engl J Med, 352 (2005) 987-996.
[15] N. J. Abbott, L. Ronnback, E. Hansson, Astrocyte-endothelial interactions at the blood-brain barrier, Nat Rev Neurosci, 7 (2006) 41-53.
[16] R. K. Oberoi, K. E. Parrish, T. T. Sio, R. K. Mittapalli, W. F. Elmquist, J. N. Sarkaria, Strategies to improve delivery of anticancer drugs across the blood-brain barrier to treat glioblastoma, Neuro-Oncology, (2015).
[17] S. Bhaskar, F. Tian, T. Stoeger, W. Kreyling, J. M. de la Fuente, V. Grazu, P. Borm, G. Estrada, V. Ntziachristos, D. Razansky, Multifunctional Nanocarriers for diagnostics, drug delivery and targeted treatment across blood-brain barrier: perspectives on tracking and neuroimaging, Particle and Fibre Toxicology, 7 (2010).
[18] I. Wilhelm, C. Fazakas, I. A. Krizbai, In vitro models of the blood-brain barrier, Acta Neurobiologiae Experimentalis, 71 (2011) 113-128.
[19] H. Yang, Nanoparticle-Mediated Brain-Specific Drug Delivery, Imaging, and Diagnosis, Pharmaceutical Research, 27 (2010) 1759-1771.
[20] X. Y. Xu, X. F. Zhang, X. H. Wang, Y. X. Li, X. B. Jing, Comparative study of paclitaxel physically encapsulated in and chemically conjugated with PEG-PLA, Polym Advan Technol, 20 (2009) 843-848.
[21] A. Zensi, D. Begley, C. Pontikis, C. Legros, L. Mihoreanu, C. Buechel, J. Kreuter, Human serum albumin nanoparticles modified with apolipoprotein A-I cross the blood-brain barrier and enter the rodent brain, Journal of Drug Targeting, 18 (2010) 842-848.
[22] A. Zensi, D. Begley, C. Pontikis, C. Legros, L. Mihoreanu, S. Wagner, C. Buechel, H. von Briesen, J. Kreuter, Albumin nanoparticles targeted with Apo E enter the CNS by transcytosis and are delivered to neurones, Journal of Controlled Release, 137 (2009) 78-86.
[23] I. Miladi, G. Le Duc, D. Kryza, A. Berniard, P. Mowat, S. Roux, J. Taleb, P. Bonazza, P. Perriat, F. Lux, O. Tillement, C. Billotey, M. Janier, Biodistribution of ultra small gadolinium-based nanoparticles as theranostic agent: Application to brain tumors, J Biomater Appl, 28 (2013) 385-394.
[24] N. T. Huynh, M. Morille, J. Bejaud, P. Legras, A. Vessieres, G. Jaouen, J. P. Benoit, C. Passirani, Treatment of 9 L Gliosarcoma in Rats by Ferrociphenol-Loaded Lipid Nanocapsules Based on a Passive Targeting Strategy via the EPR Effect, Pharmaceutical Research, 28 (2011) 3189-3198.
[25] S. C. J. Steiniger, J. Kreuter, A. S. Khalansky, I. N. Skidan, A. I. Bobruskin, Z. S. Smirnova, S. E. Severin, R. Uhl, M. Kock, K. D. Geiger, S. E. Gelperina, Chemotherapy of glioblastoma in rats using doxorubicin-loaded nanoparticles, Int J Cancer, 109 (2004) 759-767.
[26] Y. Yamashita, R. Saito, M. T. Krauze, T. Kawaguchi, C. Noble, D. C. Drummond, D. B. Kirpotin, J. W. Park, M. S. Berger, K. S. Bankiewicz, Convection-enhanced delivery of liposomal doxorubicin in intracranial brain tumor xenografts, Target Oncol, 1 (2006) 79-85.
[27] I. Brigger, J. Morizet, G. Aubert, H. Chacun, M. J. Terrier-Lacombe, P. Couvreur, G. Vassal, Poly(ethylene glycol)-coated hexadecylcyanoacrylate nanospheres display a combined effect for brain tumor targeting, J Pharmacol Exp Ther, 303 (2002) 928-936.
[28] H. Cabral, Y. Matsumoto, K. Mizuno, Q. Chen, M. Murakami, M. Kimura, Y. Terada, M. R. Kano, K. Miyazono, M. Uesaka, N. Nishiyama, K. Kataoka, Accumulation of sub-100 nm polymeric micelles in poorly permeable tumours depends on size, Nat Nanotechnol, 6 (2011) 815-823.
[29] M. Longmire, P. L. Choyke, H. Kobayashi, Clearance properties of nano-sized particles and molecules as imaging agents: considerations and caveats, Nanomedicine-Uk, 3 (2008) 703-717.
[30] M. Horowitz, R. Blasberg, P. Molnar, J. Strong, P. Kornblith, R. Pleasants, J. Fenstermacher, Regional [Misonidazole-C-14 Distribution in Experimental Rt-9-Brain Tumors, Cancer Res, 43 (1983) 3800-3807.
[31] V. A. Levin, M. Freemandove, H. D. Landahl, Permeability Characteristics of Brain Adjacent to Tumors in Rats, Arch Neurol-Chicago, 32 (1975) 785-791.
[32] R. K. Oberoi, R. K. Mittapalli, W. F. Elmquist, Pharmacokinetic Assessment of Efflux Transport in Sunitinib Distribution to the Brain, J Pharmacol Exp Ther, 347 (2013) 755-764.
[33] S. K. Hobbs, W. L. Monsky, F. Yuan, W. G. Roberts, L. Griffith, V. P. Torchilin, R. K. Jain, Regulation of transport pathways in tumor vessels: Role of tumor type and microenvironment, P Natl Acad Sci USA, 95 (1998) 4607-4612.
[34] A. I. Minchinton, I. F. Tannock, Drug penetration in solid tumours, Nat Rev Cancer, 6 (2006) 583-592.
[35] Z. Popovic, W. H. Liu, V. P. Chauhan, J. Lee, C. Wong, A. B. Greytak, N. Insin, D. G. Nocera, D. Fukumura, R. K. Jain, M. G. Bawendi, A Nanoparticle Size Series for In Vivo Fluorescence Imaging, Angew Chem Int Edit, 49 (2010) 8649-8652.
[36] M. E. Fox, F. C. Szoka, J. M. J. Frechet, Soluble Polymer Carriers for the Treatment of Cancer: The Importance of Molecular Architecture, Accounts Chem Res, 42 (2009) 1141-1151.
[37] N. Nasongkla, B. Chen, N. Macaraeg, M. E. Fox, J. M. J. Frechet, F. C. Szoka, Dependence of Pharmacokinetics and Biodistribution on Polymer Architecture: Effect of Cyclic versus Linear Polymers, J Am Chem Soc, 131 (2009) 3842-+.
[38] H. L. Kim, S. B. Lee, H. J. Jeong, D. W. Kim, Enhanced tumor targetability of PEGylated mesoporous silica nanoparticles on in vivo optical imaging according to their size, Rsc Advances, 4 (2014) 31318-31322.
[39] J. W. Seo, H. Zhang, D. L. Kukis, C. F. Meares, K. W. Ferrara, A Novel Method to Label Preformed Liposomes with ⁶⁴Cu Positron Emission Tomography (PET) Imaging, Bioconjugate Chem., 19 (2008) 2577-2584.
[40] H. Dong, N. Dube, J. Y. Shu, J. W. Seo, L. M. Mahakian, K. W. Ferrara, T. Xu, Long-Circulating 15 nm Micelles Based on Amphiphilic 3-Helix Peptide-PEG Conjugates, Acs Nano, 6 (2012) 5320-5329.
[41] H. Dong, J. Y. Shu, N. Dube, Y. Ma, M. V. Tirrell, K. H. Downing, T. Xu, 3-Helix Micelles Stabilized by Polymer Springs, Journal of the American Chemical Society, 134 (2012) 11807-11814.
[42] N. Dube, J. Y. Shu, H. Dong, J. W. Seo, E. Ingham, A. Kheirolomoom, P.-Y. Chen, J. Forsayeth, K. Bankiewicz, K. W. Ferrara, T. Xu, Evaluation of Doxorubicin-Loaded 3-Helix Micelles as Nanocarriers, Biomacromolecules, 14 (2013) 3697-3705.
[43] N. Dube, J. W. Seo, H. Dong, J. Y. Shu, R. Lund, L. M. Mahakian, K. W. Ferrara, T. Xu, Effect of Alkyl Length of Peptide-Polymer Amphiphile on Cargo Encapsulation Stability and Pharmacokinetics of 3-Helix Micelles, Biomacromolecules, 15 (2014) 2963-2970.
[44] H. Dong, R. Lund, T. Xu, Micelle Stabilization via Entropic Repulsion: Balance of Force Directionality and Geometric Packing of Subunit, Biomacromolecules, 16 (2015) 743-747.
[45] N. L. Ogihara, M. S. Weiss, D. Eisenberg, W. F. Degrado, The crystal structure of the designed trimeric coiled coil coil-VaLd: Implications for engineering crystals and supramolecular assemblies, Protein Science, 6 (1997) 80-88.
[46] J. Y. Shu, C. Tan, W. F. DeGrado, T. Xu, New design of helix bundle peptide-polymer conjugates, Biomacromolecules, 9 (2008) 2111-2117.
[47] H. B. Lee, M. D. Blaufox, Blood-Volume in the Rat, J Nucl Med, 26 (1985) 72-76.
[48] H. J. Aronen, I. E. Gazit, D. N. Louis, B. R. Buchbinder, F. S. Pardo, R. M. Weisskoff, G. R. Harsh, G. R. Cosgrove, E. F. Halpern, F. H. Hochberg, B. R. Rosen, Cerebral Blood-Volume Maps of Gliomas—Comparison with Tumor Grade and Histologic-Findings, Radiology, 191 (1994) 41-51.
[49] C. F. vanDijke, R. C. Brasch, T. P. L. Roberts, N. Weidner, A. Mathur, D. M. Shames, J. S. Mann, F. Demsar, P. Lang, H. C. Schwickert, Mammary carcinoma model: Correlation of macromolecular contrast-enhanced MR imaging characterizations of tumor microvasculature and histologic capillary density, Radiology, 198 (1996) 813-818.
[50] J. Dennie, J. B. Mandeville, J. L. Boxerman, S. D. Packard, B. R. Rosen, R. M. Weisskoff, NMR imaging of changes in vascular morphology due to tumor angiogenesis, Magnet Reson Med, 40 (1998) 793-799.
[51] C. Engvall, E. Ryding, R. Wirestam, S. Holtas, K. Ljunggren, T. Ohlsson, P. Reinstrup, Human cerebral blood volume (CBV) measured by dynamic susceptibility contrast MRI and Tc-99m-RBC SPECT, J Neurosurg Anesth, 20 (2008) 41-44.
[52] M. I. Koukourakis, S. Koukouraki, I. Fezoulidis, N. Kelekis, G. Kyrias, S. Archimandritis, N. Karkavitsas, High intratumoural accumulation of stealth (R) liposomal doxorubicin (Caelyx (R)) in glioblastomas and in metastatic brain tumours, Brit J Cancer, 83 (2000) 1281-1286.
[53] D. R. Senger, L. F. Brown, K. P. Claffey, H. F. Dvorak, Vascular-Permeability Factor, Tumor Angiogenesis and Stroma Generation, Invas Metast, 14 (1994) 385-394.
[54] J. W. Seo, L. M. Mahakian, A. Kheirolomoom, H. Zhang, C. F. Meares, R. Ferdani, C. J. Anderson, K. W. Ferrara, Liposomal Cu-64 labeling method using bifunctional chelators: poly(ethylene glycol) spacer and chelator effects, Bioconjugate Chem., 21 (2010) 1206-1215.
[55] E. T. M. Dams, P. Laverman, W. J. G. Oyen, G. Storm, G. L. Scherphof, J. W. M. Van der Meer, F. H. M. Corstens, O. C. Boerman, Accelerated blood clearance and altered biodistribution of repeated injections of sterically stabilized liposomes, J Pharmacol Exp Ther, 292 (2000) 1071-1079.
[56] C. Ricard, F. Stanchi, T. Rodriguez, M. C. Amoureux, G. Rougon, F. Debarbieux, Dynamic Quantitative Intravital Imaging of Glioblastoma Progression Reveals a Lack of Correlation between Tumor Growth and Blood Vessel Density, Plos One, 8 (2013) 15.
[57] B. R. Deane, P. L. Lantos, The Vasculature of Experimental Brain-Tumors 0.1. A Sequential Light and Electron-Microscope Study of Angiogenesis, J Neurol Sci, 49 (1981) 55-66.
[58] N. Nghi, J. Montagnese, L. R. Rogers, A. Sher, L. Wolansky, Positron Emission Tomography-Magnetic Resonance Imaging in the Evaluation of Brain Tumors: Current Status and Future Prospects, Seminars in Roentgenology, 49 (2014) 275-289.
[59] B. Ertl-Wagner, M. Ingrisch, M. Niyazi, O. Schnell, N. Jansen, S. Forster, C. la Fougere, PET-MR in patients with glioblastoma multiforme, Radiologe, 53 (2013) 682-690.

What is claimed is:

1. A conjugate comprising:
a first peptide having from about 10 to about 100 amino acids, wherein the peptide adopts a helical structure;
a first polymer comprising a hydrophilic polymer covalently linked to an amino acid residue of the peptide, other than the N-terminal and C-terminal amino acid residues;
a metal binding ligand covalently linked to the C-terminal amino acid residue of the peptide; and
a hydrophobic moiety covalently linked to the N-terminus of the peptide, wherein the hydrophobic moiety comprises a third polymer or a lipid moiety.

2. The conjugate of claim 1, wherein the peptide is selected from the group consisting of SEQ ID NO: 1, SEQ ID NO: 2, SEQ ID NO: 4, SEQ ID NO:5 and SEQ ID NO: 6.

3. The conjugate of claim 1, wherein the first polymer comprises polyethylene glycol.

4. The conjugate of claim 1, wherein the metal binding ligand is a copper binding ligand.

5. The conjugate of claim 4, wherein the copper binding ligand comprises 6-aminobenzyl TETA.

6. The conjugate of claim 1, wherein
the first peptide comprises SEQ ID NO:1;
the first polymer comprises polyethylene glycol with a molecular weight of about 2000 Da;
the metal binding ligand comprises 6-aminobenzyl TETA; and
the hydrophobic moiety comprises the lipid moiety which comprises lysine and two C₁₈ acyl chains.

7. A particle comprising from about 20 to about 200 conjugates, wherein each conjugate comprises:
a first peptide having from about 10 to about 100 amino acids, wherein the peptide adopts a helical structure;
a first polymer comprising a hydrophilic polymer covalently linked to an amino acid residue of the peptide, other than the N-terminal and C-terminal amino acid residues;
a metal binding ligand or a second polymer covalently linked to the C-terminal amino acid residue of the peptide; and
a hydrophobic moiety covalently linked to the N-terminus of the peptide, wherein the hydrophobic moiety comprises a third polymer or a lipid moiety,
such that at least one conjugate includes the metal binding ligand covalently linked to the C-terminal amino acid residue of the peptide.

8. The particle of claim 7, further comprising at least one additional agent, each independently selected from the group consisting of a therapeutic agent, a diagnostic agent, DNA, and an oligonucleotide.

9. A method of delivering a therapeutic or diagnostic agent to a brain tumor in a subject in need thereof, comprising:
administering to the subject a composition comprising a particle of claim 7 and an effective amount of the therapeutic or diagnostic agent, thereby delivering the therapeutic or diagnostic agent to the brain tumor.

10. The method of claim 9, wherein the brain tumor is a glioblastoma multiforme.

11. The method of claim 9, wherein the therapeutic agent is selected from the group consisting of temozolomide, doxorubicin, paclitaxel, and rapamycin.

12. A method of treating cancer comprising administering to a subject in need thereof, a therapeutically effective amount of a particle of claim 7 and at least one therapeutic agent, thereby treating cancer.

13. The method of claim 12, wherein the at least one therapeutic agent is doxorubicin.

14. A method of visualizing tissue in a subject, the method comprising
administering to the subject an effective amount of a particle of claim 7 comprising ⁶⁴Cu;
imaging the tissue using PET; and
obtaining at least one image of tissue from the subject using the particle, thereby visualizing the tissue.

15. The method of claim 14, further comprising obtaining the image during administration, after administration, or both during and after administration of the particle.
